(19) United States
(12) Patent Application Publication
(10) Pub. No.: US 2026/0335309 A1
(43) Pub. Date: Oct. 29, 2026

(54) METHODS OF TREATING UVEAL MELANOMA

(71) Applicants: IDEAYA BIOSCIENCES, INC., South San Francisco, CA (US); PFIZER INC., New York, NY (US)

(72) Inventors: MATTHEW ANTHONY MAURER, South San Francisco, CA (US); MICHAEL GABRIEL O'QUIGLEY, South San Francisco, CA (US); JULIE HAMBLETON, South San Francisco, CA (US)

(21) Appl. No.: 19/106,914

(22) PCT Filed: Aug. 25, 2023
(86) PCT No.: PCT/US2023/072954

§ 371 Date: Feb. 26, 2025

Related U.S. Application Data
(60) Provisional application No. 63/373,687, filed on Aug. 26, 2022.
(60) Provisional application No. 63/375,155, filed on Sep. 9, 2022.
(60) Provisional application No. 63/386,858, filed on Dec. 9, 2022.
(60) Provisional application No. 63/497,363, filed on Apr. 20, 2023.

(51) Int. Cl. A61K 31/497; A61K 31/4545; A61P 35/00

(52) U.S. Cl. CPC A61K 31/497; A61K 31/4545; A61P 35/00

(57) ABSTRACT

Provided herein is a combination therapy and methods of using such combination therapy as a neoadjuvant therapy and/or an adjuvant therapy in uveal melanoma, inter alia to reduce the risk of metastasis and/or enucleation, and/or preserving eye function. Uveal melanoma (UM) is the most common primary intraocular malignant tumor in adults. The protein kinase C (PKC) inhibitor, sotrastaurin, has been shown to have activity against certain PKC isototypes and has been shown to selectively inhibit the growth of uveal melanoma cells harboring GNAQ mutations by targeting PKC/ERK1/2 and PKC/NF-xB pathways.

### RELATED APPLICATIONS

This application claims priority to U.S. Provisional Application No. 63/373,687, filed on Aug. 26, 2022; U.S. Provisional Application No. 63/375,155, filed on Sep. 9, 2022; U.S. Provisional Application No. 63/386,858, filed on Dec. 9, 2022; and U.S. Provisional Application No. 63/497,363, filed on Apr. 20, 2023. The content of each application is incorporated by reference in their entirety.

### BACKGROUND

Uveal melanoma (UM) is the most common primary intraocular malignant tumor in adults. One protein kinase C (PKC) inhibitor, sotrastaurin, has been shown to have activity against certain PKC isototypes and has been shown to selectively inhibit the growth of uveal melanoma cells harboring GNAQ mutations by targeting PKC/ERK1/2 and PKC/NF-xB pathways (see X. Wu, et al., Mol. Cancer Ther., Vol. 11, pages 1905-1914, 2012). There still remains an unmet need to provide next generation PKC inhibitors for treating uveal melanoma that have improved efficacy at lower dosage amounts to achieve tumor regression, improved potency, hERG activity, absorption, gastrointestinal tolerance and kinase selectivity. PCT application no. PCT/IB2015/055951 (WO 2016/020864) discloses a number of potent and selective PKC inhibitors.

The c-MET receptor has been shown to be expressed in a number of human cancers. c-Met and its ligand, HGF, have also been shown to be co-expressed at elevated levels in a variety of human cancers (particularly sarcomas). However, because the receptor and ligand are usually expressed by different cell types, c-MET signaling is most commonly regulated by tumor-stroma (tumor-host) interactions. Furthermore, c-MET gene amplification, mutation, and rearrangement have been observed in a subset of human cancers. Families with germline mutations that activate c-MET kinase are prone to multiple kidney tumors as well as tumors in other tissues. Numerous studies have correlated the expression of c-MET and/or HGF/SF with the state of disease progression of different types of cancer (including lung, colon, breast, prostate, liver, pancreas, brain, kidney, ovaries, stomach, skin, and bone cancers). Furthermore, the overexpression of c-MET or HGF have been shown to correlate with poor prognosis and disease outcome in a number of major human cancers including lung, liver, gastric, and breast. c-MET has also been directly implicated in cancers without a successful treatment regimen such as pancreatic cancer, glioma, and hepatocellular carcinoma. PCT application no.

PCT application no. PCT/IB2005/002837 (WO 2006/021884) discloses a number of potent and selective c-MET inhibitors. PCT application no. PCT/IB2015/055951 (WO 2016/020864) discloses a number of potent and selective PKC inhibitors.

Provided herein is a tyrosine-kinase targeted therapy combination to address some of the issues discussed herein.

### SUMMARY

Provided herein is a neoadjuvant therapy comprising administering a PKC inhibitor and a c-MET inhibitor. Also provided herein is a primary therapy for treatment of uveal melanoma comprising administering a PKC inhibitor and a c-MET inhibitor. The neoadjuvant therapy and the primary therapy are, in each case, useful for the treatment of uveal melanoma, including ocular tumors. In one aspect, the ocular tumors are intraocular tumors. In another aspect, a PKC inhibitor and a c-MET inhibitor is also useful as an adjuvant therapy. A neoadjuvant therapy can be administered in advance of or during an interventional procedure, which can be a primary interventional procedure. An adjuvant therapy can be used during or after an interventional procedure, which can be a primary interventional procedure. In each such case, a (primary) interventional procedure can include, for example, irradiation of the ocular tumor or surgical intervention (e.g., resection or removal) of the ocular tumor.

Thus, in one aspect, provided herein is a method of treating uveal melanoma in a patient having an ocular tumor, the method comprising a) administering a PKC inhibitor and administering a c-MET inhibitor to the patient as a neoadjuvant therapy; b) reducing the size of the ocular tumor by at least 10%; and c) treating the reduced-size ocular tumor with an interventional procedure.

In another aspect, provided herein is a method of treating a patient having uveal melanoma with an ocular tumor with a size indicated for an interventional procedure comprising irradiating the ocular tumor, the method comprising a) administering a PKC inhibitor and administering a c-MET inhibitor to the patient as a neoadjuvant therapy; b) reducing the size of the ocular tumor by at least 10%; and c) treating the reduced-size ocular tumor with an interventional procedure other than enucleation, including for example with an interventional procedure comprising irradiation.

In another aspect, provided herein is a method of treating a patient having uveal melanoma with an ocular tumor with a size indicated for an interventional procedure comprising enucleation of the eye which is the situs of the ocular tumor, the method comprising a) administering a PKC inhibitor and administering a c-MET inhibitor to the patient as a neoadjuvant therapy; b) reducing the size of the ocular tumor by at least 10%; and c) treating the reduced-size ocular tumor with an interventional procedure other than enucleation.

In each of these such aforementioned aspects of the invention, the method of treatment can comprise administering a PKC inhibitor and administering a c-MET inhibitor to the patient as a neoadjuvant therapy whereby the size of the ocular tumor by at least 10%.

In various other aspects, as described in more detail below, the method of treatment of uveal melanoma can comprise administering a PKC inhibitor and administering a c-MET inhibitor to the patient as a neoadjuvant therapy to reduce the size of an ocular tumor to enable an alternative treatment paradigm. For example, and without limitation except as explicitly claimed, neoadjuvant treatment with a PKC inhibitor and a c-MET inhibitor can reduce a large size ocular tumor to either a medium size ocular tumor or a small size ocular tumor, to enable an interventional procedure other than enucleation (e.g. for a medium size ocular tumor) or to enable an observational period (e.g., for a small size ocular tumor) prior to or in lieu of an interventional procedure. As another example, and without limitation except as explicitly claimed, neoadjuvant treatment with a PKC inhibitor and a c-MET inhibitor can reduce a medium size ocular tumor to be a small size ocular tumor, to enable an interventional procedure other than enucleation, such as an alternative radiotherapy approach than otherwise would have been applicable for the original medium size ocular tumor or to enable an to enable an observational period prior to or in lieu of an interventional procedure.

In each of such aspects of the invention, an ocular tumor size can be defined by a set of dimensions known in the art. Such dimensions can include for example, a basal diameter, typically measured a longest basal diameter (LBD) of the ocular tumor and/or an apical height. Another feature of an ocular tumor that can impact selection of an interventional procedure is the relative proximity of the location or situs of the ocular tumor to the ocular nerve. Accordingly, various such aspects of the invention are described herein, including in connection with certain such dimensions as defined by guidelines issued by regulatory agencies or other governmental or private authoritative bodies, such as the National Comprehensive Cancer Network (NCCN) Guidelines Version 2.2022 (Uveal Melanoma), National Cancer Institute (NCI) clinical practice guidelines, among others known in the art, and/or by standard of care practices developed by ocular oncologists or ocular surgeons informed of such guidelines and practicing in the field of treating uveal melanoma.

In another aspect, provided herein is a method of treating uveal melanoma in a patient having an ocular tumor with a size defined by a set of dimensions comprising (i) greater than 16 mm LBD and any apical height; (ii) any LBD and greater than 8 mm apical height; or (iii) less than 16 mm LBD and 3-8 mm apical height, the method comprising a) administering a PKC inhibitor and administering a c-MET inhibitor to the patient as a neoadjuvant therapy; b) reducing the size of the ocular tumor to dimensions of less than 5 mm LBD and less than 3 mm apical height; and optionally c) treating the reduced-size ocular tumor with an interventional procedure. Without limitation except as explicitly claimed, such aspect can reflect reducing a large size ocular tumor, e.g., with dimensions as defined as in (i) or (ii), or a medium size tumor, e.g., with dimensions as defined as in (iii), in each case to a small size ocular tumor. Such a small size ocular tumor may be monitored during an observational period prior to or in lieu of an interventional procedure.

In yet another aspect, provided herein is a method of treating uveal melanoma in a patient having an ocular tumor with a size defined by a set of dimensions comprising (i) greater than 16 mm LBD and any apical height, or (ii) any LBD and greater than 8 mm apical height, the method comprising a) administering a PKC inhibitor and administering a c-MET inhibitor to the patient as a neoadjuvant therapy; b) reducing the size of the ocular tumor to dimensions of less than 16 mm LBD and less than 8 mm apical height; and optionally c) treating the reduced-size ocular tumor with an interventional procedure. Without limitation except as explicitly claimed, such aspect can reflect reducing a large size ocular tumor, e.g., with dimensions as defined as in (i) or (ii), in each case to a medium size ocular tumor or a small size ocular tumor.

In the immediately preceding aspect, for example, for a patient having an ocular tumor with a size defined by a set of dimensions comprising (i) greater than 16 mm LBD and any apical height or (ii) any LBD and greater than 8 mm apical height, the method of treating uveal melanoma can comprise a) administering a PKC inhibitor and a c-MET inhibitor to the patient as a neoadjuvant therapy; b) reducing the size of the ocular tumor to dimensions of less than 16 mm LBD and 3-8 mm apical height; and c) treating the reduced-size ocular tumor with an interventional procedure. Without limitation except as explicitly claimed, this example can reflect reducing a large size ocular tumor, e.g., with dimensions as defined as in (i) or (ii), in each case to a medium size ocular tumor.

In still another aspect, provided herein is a method of treating uveal melanoma in a patient having an ocular tumor with a size defined by a set of dimensions comprising (i) greater than 19 mm LBD and any apical height, (ii) any LBD and greater than 10 mm apical height, (iii) any LBD, greater than 8 mm apical height, and the ocular tumor is located close to, including proximal to or adjacent, the optic nerve, or (iv) less than or equal to 19 mm LBD and 2.5-10 mm apical height, the method comprising a) administering a PKC inhibitor and a c-MET inhibitor as a neoadjuvant therapy; b) reducing the size of the ocular tumor to dimensions of 5-19 mm LBD or less than 2.5 mm apical height; and c) treating the reduced-size ocular tumor with an interventional procedure. Preferably, step (b) of this aspect comprises (b) reducing the size of the ocular tumor to dimensions of 5-19 mm LBD and less than 2.5 mm apical height. Without limitation except as explicitly claimed, this example can reflect reducing a large size ocular tumor, e.g., with dimensions as defined as in (i), (ii), (iii) or (iv), in each case to a medium size ocular tumor or a small size ocular tumor.

In an aspect, provided herein is a method of treating uveal melanoma in a patient having an ocular tumor with a size defined by a set of dimensions comprising (i) greater than 19 mm LBD and any apical height, (ii) any LBD and greater than 10 mm apical height, or (iii) greater than 8 mm apical height, any LBD, and the ocular tumor is located close to, including proximal to or adjacent, the optic nerve, the method comprising a) administering a PKC inhibitor and administering a c-MET inhibitor to the patient as a neoadjuvant therapy; b) reducing the size of the ocular tumor to dimensions of less than or equal to 19 mm LBD and 2.5-10 mm apical height; and c) treating the reduced-size ocular tumor with an interventional procedure. Without limitation except as explicitly claimed, this example can reflect reducing a large size ocular tumor, e.g., with dimensions as defined as in (i), (ii), (iii), in each case to a medium size ocular tumor or a small size ocular tumor.

### DETAILED DESCRIPTION

Provided herein is a combination therapy comprising a PKC inhibitor, or a pharmaceutically acceptable salt thereof, and c-MET inhibitor, or a pharmaceutically acceptable salt thereof. The combination therapy is useful as a neoadjuvant therapy for the treatment of uveal melanoma including ocular tumors. The combination therapy is also useful as an adjuvant therapy.

### DEFINITIONS

Listed below are definitions of various terms used herein. These definitions apply to the terms as they are used throughout this specification and claims, unless otherwise limited in specific instances, either individually or as part of a larger group.

Unless defined otherwise, all technical and scientific terms used herein generally have the same meaning as commonly understood by one of ordinary skill in the art. Generally, the nomenclature used herein and the laboratory procedures in cell culture, molecular genetics, organic chemistry, and peptide chemistry are those well-known and commonly employed in the art.

As used herein, the articles “a” and “an” refer to one or to more than one (i.e., to at least one) of the grammatical object of the article. By way of example, “an element” means one element or more than one element. Furthermore, use of the term “including” as well as other forms, such as “include,” “includes,” and “included,” is not limiting.

As used herein, the term “about” will be understood by persons of ordinary skill in the art and will vary to some extent on the context in which it is used. As used herein when referring to a measurable value such as an amount, a temporal duration, and the like, the term “about” is meant to encompass variations of ±20% or ±10%, including±5%, ±1%, and ±0.1% from the specified value, as such variations are appropriate to perform the disclosed methods. For example, a dose of about 300 mg may be understood to mean that the dose may vary between 270 mg and 330 mg.

As used in the specification and in the claims, the term “comprising” may include the embodiments “consisting of” and “consisting essentially of.” The terms “comprise(s),” “include(s),” “having,” “has,” “may,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated compounds, which allows the presence of only the named compounds, along with any pharmaceutically acceptable carriers, and excludes other compounds.

“Alkyl” means a linear saturated monovalent hydrocarbon radical of one to six carbon atoms or a branched saturated monovalent hydrocarbon radical of three to six carbon atoms, e.g., methyl, ethyl, propyl, 2-propyl, butyl, pentyl, and the like. It will be recognized by a person skilled in the art that the term “alkyl” may include “alkylene” groups.

“Alkylene” means a linear saturated divalent hydrocarbon radical of one to six carbon atoms or a branched saturated divalent hydrocarbon radical of three to six carbon atoms unless otherwise stated e.g., methylene, ethylene, propylene, 1-methylpropylene, 2-methylpropylene, butylene, pentylene, and the like.

“Alkenyl” means a linear monovalent hydrocarbon radical of two to six carbon atoms or a branched monovalent hydrocarbon radical of three to six carbon atoms containing a double bond, e.g., propenyl, butenyl, and the like.

“Alkynyl” means a linear monovalent hydrocarbon radical of two to six carbon atoms or a branched monovalent hydrocarbon radical of three to six carbon atoms containing a triple bond, e.g., ethynyl, propynyl, butynyl, and the like.

“Alkoxy” means a —OR radical where R is alkyl as defined above, e.g., methoxy, ethoxy, propoxy, or 2-propoxy, n-, iso-, or tert-butoxy, and the like.

“Aryl” means a monovalent monocyclic or bicyclic aromatic hydrocarbon radical of 6 to 10 ring atoms e.g., phenyl or naphthyl.

“Cycloalkyl” means a monocyclic monovalent hydrocarbon radical of three to six carbon atoms which may be saturated or contains one double bond. Cycloalkyl may be unsubstituted or substituted with one or two substituents independently selected from alkyl, halo, alkoxy, hydroxy, or cyano. Examples include, but are not limited to, cyclopropyl, cyclobutyl, cyclopentyl, cyclohexyl, 1-cyanocycloprop-1-yl, 1-cyanomethylcycloprop-1-yl, 3-fluorocyclohexyl, and the like. When cycloalkyl contains a double bond, it may be referred to herein as cycloalkenyl.

“Halo” means fluoro, chloro, bromo, or iodo, preferably fluoro or chloro.

“Haloalkyl” means alkyl radical as defined above, which is substituted with one to five halogen atoms, such as fluorine or chlorine, including those substituted with different halogens, e.g., —CH₂Cl, —CF₃, —CHF₂, —CH₂CF₃, —CF₂CF₃, —CF(CH₃)₂, and the like. When the alkyl is substituted with only fluoro, it can be referred to as fluoroalkyl.

“Haloalkoxy” means a —OR radical where R is haloalkyl as defined above e.g., —OCF₃, —OCHF₂, and the like. When R is haloalkyl where the alkyl is substituted with only fluoro, it is referred to as fluoroalkoxy.

“Heteroaryl” means a monovalent monocyclic or bicyclic aromatic radical of 5 to 10 ring atoms, unless otherwise stated, where one or more, (in one embodiment, one, two, or three), ring atoms are heteroatom selected from N, O, or S, the remaining ring atoms being carbon. Non-limiting examples of heteroaryl groups include pyridyl, pyridazinyl, pyrazinyl, pyrimindinyl, triazinyl, quinolinyl, quinoxalinyl, quinazolinyl, cinnolinyl, phthalazinyl, benzotriazinyl, purinyl, benzimidazolyl, benzopyrazolyl, benzotriazolyl, benzisoxazolyl, isobenzofuryl, isoindolyl, indolizinyl, benzotriazinyl, thienopyridinyl, thienopyrimidinyl, pyrazolopyrimidinyl, imidazopyridines, benzothiaxolyl, benzofuranyl, benzothienyl, indolyl, quinolyl, isoquinolyl, isothiazolyl, pyrazolyl, indazolyl, pteridinyl, imidazolyl, triazolyl, tetrazolyl, oxazolyl, isoxazolyl, thiadiazolyl, pyrrolyl, thiazolyl, furyl, thienyl, and the like. As defined herein, the terms “heteroaryl” and “aryl” are mutually exclusive. When the heteroaryl ring contains 5- or 6 ring atoms it is also referred to herein as 5- or 6-membered heteroaryl.

“Heterocyclyl” or “heterocycloalkyl” means a saturated or unsaturated monovalent monocyclic group of 4 to 8 ring atoms in which one or two ring atoms are heteroatom selected from N, O, or S(O)ₙ, where n is an integer from 0 to 2, the remaining ring atoms being C. Additionally, one or two ring carbon atoms in the heterocyclyl ring can optionally be replaced by a —CO-group. More specifically the term heterocyclyl includes, but is not limited to, azetidinyl, oxetanyl, pyrrolidino, piperidino, homopiperidino, 2-oxopyrrolidinyl, 2-oxopiperidinyl, morpholino, piperazino, tetrahydro-pyranyl, thiomorpholino, and the like. When the heterocyclyl ring is unsaturated it can contain one or two ring double bonds provided that the ring is not aromatic. When heterocyclyl contains at least one nitrogen atom, it may be referred to herein as heterocycloamino.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a dose range of “200 mg to about 600 mg” should be interpreted to include not only the explicitly recited concentration of about 200 mg to about 600 mg, but also include individual dosage (e.g., 250 mg, 400 mg, 550 mg) and the sub-ranges (e.g., 250 mg to 450 mg) within the indicated range. To further illustrate, a tumor size reduction of “30%-50%” should be interpreted to include not only the explicitly recited concentration of about 30% to about 50%, but also include individual percentages (e.g., 35%, 40%, 50%) and the sub-ranges (e.g., 35%-45%) within the indicated range. The term “about” can include ±1%, ±2%, ±3%, ±4%, ±5%, ±6%, ±7%, ±8%, ±9%, or ±10%, of the numerical value(s) being modified. In addition, the phrase “about ‘x’ to ‘y’” includes “about ‘x’ to about ‘y’”.

The terms “combination,” “therapeutic combination,” “pharmaceutical combination,” or “combination product” as used herein refer to either a fixed combination in one dosage unit form, or non-fixed combination in separate dosage forms, or a kit of parts for the combined administration where two or more therapeutic agents may be administered independently, at the same time or separately within time intervals.

As used herein, the term “non-fixed combination” means that the active ingredients, e.g., Compound 1 and Compound 2, are both administered to a patient as separate entities either simultaneously, concurrently or sequentially, wherein such administration provides therapeutically effective levels of the two compounds in the body of the patient.

The term “combination therapy” refers to the administration of two or more therapeutic agents to treat a therapeutic condition or disorder described in the present disclosure. Such administration encompasses co-administration of these therapeutic agents in a substantially simultaneous manner, such as in a single formulation having a fixed ratio of active ingredients or in separate formulations (i.e., in separate dosage units, for example, separate tablets, capsules and/or intravenous formulations) for each active ingredient. In addition, such administration also encompasses use of each type of therapeutic agent in a sequential or separate manner, either at approximately the same time or at different times. Regardless of whether the active ingredients are administered as a single formulation or in separate formulations, the agents are administered to the same patient as part of the same course of therapy. The agents may be administered at the same point in time or immediately following one another. The agents may be administered at any order. The agents may be administered separately at different times during the course of therapy in such time intervals that the combination therapy is effective in treating cancer. In any case, the treatment regimen will provide beneficial effects in treating the conditions or disorders described herein.

As used herein, the term “free base equivalent” refers to the amount of the active agent (e.g., Compound 1 or Compound 2) present in the active agent or pharmaceutically acceptable salt thereof. Stated alternatively, the term “free base equivalent” means either an amount of Compound 1 or Compound 2 free base, or the equivalent amount of Compound 1 or Compound 2 free base that is provided by a salt of said compound.

As used herein, “metastasis” or “metastatic” is meant the spread of cancer from its primary site to other places in the body. Cancer cells can break away from a primary tumor, penetrate into lymphatic and blood vessels, circulate through the bloodstream, and grow in a distant focus (metastasize) in normal tissues elsewhere in the body. Metastasis can be local or distant. Metastasis is a sequential process, contingent on tumor cells breaking off from the primary tumor, traveling through the bloodstream, and stopping at a distant site. At the new site, the cells establish a blood supply and can grow to form a life-threatening mass. Both stimulatory and inhibitory molecular pathways within the tumor cell regulate this behavior, and interactions between the tumor cell and host cells in the distant site are also significant.

As used herein, the term “treating” or “treatment” refers to inhibiting a disease; for example, inhibiting a disease, condition, or disorder in an individual who is experiencing or displaying the pathology or symptomology of the disease, condition, or disorder (i.e., arresting further development of the pathology and/or symptomology) or ameliorating the disease; for example, ameliorating a disease, condition, or disorder in an individual who is experiencing or displaying the pathology or symptomology of the disease, condition, or disorder (i.e., reversing the pathology and/or symptomology) such as decreasing the severity of the disease.

As used herein, the term “prevent” or “prevention” means no disorder or disease development if none had occurred, or no further disorder or disease development if there had already been development of the disorder or disease. Also considered is the ability of one to prevent some or all of the symptoms associated with the disorder or disease.

As used herein, the term “patient,” “individual,” or “subject” refers to a human or a non-human mammal. Non-human mammals include, for example, livestock and pets, such as ovine, bovine, porcine, canine, feline and marine mammals. Preferably, the patient, subject, or individual is human.

As used herein, the term “First-Line MUM patients” refers to patients that were not given prior systemic treatment in the metastatic setting including no prior chemo-embolization, no radiation to the metastatic sites or ablation to the liver lesions.

As used herein, the terms “effective amount,” “pharmaceutically effective amount,” and “therapeutically effective amount” refer to a nontoxic but sufficient amount of an agent to provide the desired biological result. That result may be reduction or alleviation of the signs, symptoms, or causes of a disease, or any other desired alteration of a biological system. An appropriate therapeutic amount in any individual case may be determined by one of ordinary skill in the art using routine experimentation.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the term “pharmaceutically acceptable salt” refers to derivatives of the disclosed compounds wherein a parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts described herein include the conventional non-toxic salts of the parent compound formed, for example, from non-toxic inorganic or organic acids. The pharmaceutically acceptable salts discussed herein can be synthesized from the parent compound which contains a basic or acidic moiety by conventional chemical methods.

Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are used. The phrase “pharmaceutically acceptable salt” is not limited to a mono, or 1:1, salt. For example, “pharmaceutically acceptable salt” also includes bis-salts, such as a bis-hydrochloride salt. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

As used herein, the term “composition” or “pharmaceutical composition” refers to a mixture of at least one compound with a pharmaceutically acceptable carrier. The pharmaceutical composition facilitates administration of the composition to a patient or subject. Multiple techniques of administering a compound exist in the art including, but not limited to, intravenous, oral, aerosol, parenteral, ophthalmic, pulmonary, and topical administration.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound disclosed herein, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations.

As used herein, “pharmaceutically acceptable carrier” also includes any and all coatings, antibacterial and antifungal agents, and absorption delaying agents, and the like that are compatible with the activity of a compound disclosed herein, and are physiologically acceptable to the patient. Supplementary active compounds may also be incorporated into the compositions. The “pharmaceutically acceptable carrier” may further include a pharmaceutically acceptable salt of the compound(s) disclosed herein. Other additional ingredients that may be included in the pharmaceutical compositions are known in the art and described, for example, in Remington's Pharmaceutical Sciences (Genaro, Ed., Mack Publishing Co., 1985, Easton, PA), which is incorporated herein by reference.

The term “single formulation” as used herein refers to a single carrier or vehicle formulated to deliver effective amounts of both therapeutic agents to a patient. The single vehicle is designed to deliver an effective amount of each of the agents, along with any pharmaceutically acceptable carriers or excipients. In some embodiments, the vehicle is a tablet, capsule, pill, or a patch. In other embodiments, the vehicle is a solution or a suspension.

The term “unit dose” is used herein to mean simultaneous administration of both agents together, in one dosage form, to the patient being treated. In some embodiments, the unit dose is a single formulation. The term “a unit dose,” as used herein can also refer to the simultaneous administration of both agents separately, in two dosage forms, to the patient being treated. In certain embodiments, the unit dose includes one or more vehicles such that each vehicle includes an effective amount of at least one of the agents along with pharmaceutically acceptable carriers and excipients. In some embodiments, the unit dose is one or more tablets, capsules, pills, or patches administered to the patient at the same time.

The dose amounts (for Compound 1 and Compound 2) are expressed as free base equivalent amounts, unless indicated otherwise.

The combination of agents described herein may display a synergistic effect. See, for example, Wagle, M-C., et al., Preclinical evaluation of PKC and MET inhibitor combination in primary and metastatic uveal melanoma, AACR Meeting 2021, the entire content of which is hereby incorporated by reference. The term “synergistic effect” as used herein, refers to action of two agents such as, for example, Compound 1 and Compound 2, producing an effect, for example, slowing the symptomatic progression of cancer or symptoms thereof, which is greater than the simple addition of the effects of each drug administered by themselves. A synergistic effect can be calculated, for example, using suitable methods such as the Sigmoid-Emax equation (Holford, N. H. G. and Scheiner, L. B., Clin. Pharmacokinet. 6:429-453 (1981)), the equation of Loewe additivity (Loewe, S. and Muischnek, H., Arch. Exp. Pathol Pharmacol. 114:313-326 (1926)) and the median-effect equation (Chou, T. C. and Talalay, P., Adv. Enzyme Regul. 22:27-55 (1984)). Each equation referred to above can be applied to experimental data to generate a corresponding graph to aid in assessing the effects of the drug combination. The corresponding graphs associated with the equations referred to above are the concentration-effect curve, isobologram curve and combination index curve, respectively.

As used herein, the term “synergy” refers to the effect achieved when the active ingredients, i.e., Compound 1 and Compound 2, used together is greater than the sum of the effects that results from using the compounds separately.

In an embodiment, provided herein is a combination therapy comprising an effective amount of Compound 1 and Compound 2. An “effective amount” of a combination of agents (i.e., Compound 1 and Compound 2) is an amount sufficient to provide an observable improvement over the baseline clinically observable signs and symptoms of the disorders treated with the combination.

An “oral dosage form” includes a unit dosage form prescribed or intended for oral administration.

### Methods of Treatment

In an aspect, provided herein is a method of treating uveal melanoma in a patient having an ocular tumor, the method comprising
a) administering a PKC inhibitor and administering a c-MET inhibitor to the patient as a neoadjuvant therapy;
b) reducing the size of the ocular tumor by at least 10%; and
c) treating the reduced-size ocular tumor with an interventional procedure.

In an embodiment, the ocular tumor is defined by a set of dimensions comprising (i) greater than or equal to 5 mm largest basal diameter (LBD) and any apical height, or (ii) any LBD and greater than 3 mm apical height. In another embodiment, the ocular tumor is defined by being greater than or equal to 5 mm largest basal diameter (LBD) and any apical height. In yet another embodiment, the ocular tumor is defined by being any LBD and greater than 3 mm apical height.

In an aspect, provided herein is a method of treating a patient having uveal melanoma with an ocular tumor with a size indicated for an interventional procedure comprising irradiating the ocular tumor, the method comprising
a) administering a PKC inhibitor and administering a c-MET inhibitor to the patient as a neoadjuvant therapy;
b) reducing the size of the ocular tumor by at least 10%; and
c) treating the reduced-size ocular tumor with an interventional procedure other than enucleation.

In another aspect, provided herein is a method of treating uveal melanoma in a patient having an ocular tumor with a size defined by a set of dimensions comprising (i) greater than 16 mm LBD and any apical height; (ii) any LBD and greater than 8 mm apical height; or (iii) less than 16 mm LBD and 3-8 mm apical height, the method comprising
a) administering a PKC inhibitor and administering a c-MET inhibitor to the patient as a neoadjuvant therapy;
b) reducing the size of the ocular tumor to dimensions of less than 5 mm LBD and less than 3 mm apical height; and
c) treating the reduced-size ocular tumor with an interventional procedure.

In yet another aspect, provided herein is a method of treating uveal melanoma in a patient having an ocular tumor with a size defined by a set of dimensions comprising (i) greater than 16 mm LBD and any apical height; (ii) any LBD and greater than 8 mm apical height; or (iii) less than 16 mm LBD and 3-8 mm apical height, the method comprising
a) administering a PKC inhibitor and administering a c-MET inhibitor to the patient; and
b) reducing the size of the ocular tumor defined by a set of dimensions having less than 5 mm LBD and less than 3 mm apical height. In an embodiment, the method further comprising treating the reduced-size ocular tumor with an interventional procedure.

In yet another aspect, provided herein is a method of treating uveal melanoma in a patient having an ocular tumor with a size defined by a set of dimensions comprising (i) greater than 16 mm LBD and any apical height, or (ii) any LBD and greater than 8 mm apical height, the method comprising
a) administering a PKC inhibitor and administering a c-MET inhibitor to the patient as a neoadjuvant therapy;
b) reducing the size of the ocular tumor to dimensions of less than 16 mm LBD and less than 8 mm apical height; and
c) treating the reduced-size ocular tumor with an interventional procedure.

In an embodiment, the step (b) is reducing the size of the ocular tumor to dimensions of less than 16 mm LBD and 3-8 mm apical height.

In still another aspect, provided herein is a method of treating uveal melanoma in a patient having an ocular tumor with a size defined by a set of dimensions comprising (i) greater than 19 mm LBD and any apical height, (ii) any LBD and greater than 10 mm apical height, (iii) greater than 8 mm apical height, any LBD, and the tumor is located close to the optic nerve, or (iv) less than or equal to 19 mm LBD and 2.5-10 mm apical height, the method comprising
a) administering a PKC inhibitor and administering a c-MET inhibitor to the patient as a neoadjuvant therapy;
b) reducing the size of the ocular tumor defined by a set of dimensions having 5-19 mm LBD or less than 2.5 mm apical height; and
c) treating the reduced-size ocular tumor with an interventional procedure.

In yet another aspect, provided herein is a method of treating uveal melanoma in a patient having an ocular tumor, the method comprising:
a) determining a first therapeutically effective dose of radiation for treating the ocular tumor;
b) administering a PKC inhibitor and a c-MET inhibitor to the patient as a neoadjuvant therapy;
c) reducing the size of the ocular tumor by at least 10%; and
d) irradiating the reduced-size ocular tumor with a second therapeutically effective dose of radiation less than the first therapeutically effective dose of radiation.

In still another aspect, provided herein is a method of treating uveal melanoma in a patient having an ocular tumor, the method comprising:
a) determining a first therapeutically effective dose of radiation for treating the ocular tumor;
b) administering a PKC inhibitor and a c-MET inhibitor to the patient as a neoadjuvant therapy;
c) reducing the size of the ocular tumor; and
d) irradiating the reduced-size ocular tumor with a second therapeutically effective dose of radiation at least 10% less than the first therapeutically effective dose of radiation.

In an aspect, provided herein is a method of treating uveal melanoma in a patient having an ocular tumor, the method comprising:
a) determining a first therapeutically effective dose of radiation for treating the ocular tumor;
b) administering a PKC inhibitor and a c-MET inhibitor to the patient as a neoadjuvant therapy to reduce the size of the ocular tumor by at least 10%; and
c) irradiating the reduced-size ocular tumor with a second therapeutically effective dose of radiation less than the first therapeutically effective dose of radiation.

In another aspect, provided herein is a method of treating uveal melanoma in a patient having an ocular tumor, the method comprising:
a) determining a first therapeutically effective dose of radiation for treating the ocular tumor;
b) administering a PKC inhibitor and a c-MET inhibitor to the patient as a neoadjuvant therapy to reduce the size of the ocular tumor; and
c) irradiating the reduced-size ocular tumor with a second therapeutically effective dose of radiation at least 10% less than the first therapeutically effective dose of radiation.

In yet another aspect, provided herein is a method of treating uveal melanoma in a patient having an ocular tumor, the method comprising:
a) determining a first therapeutically effective dose of radiation for treating the ocular tumor;
b) administering a PKC inhibitor and a c-MET inhibitor to the patient as a neoadjuvant therapy over a period of time sufficient to reduce the size of the ocular tumor by at least 10%; and
c) irradiating the reduced-size ocular tumor with a second therapeutically effective dose of radiation less than the first therapeutically effective dose of radiation.

In still another aspect, provided herein is a method of treating uveal melanoma in a patient having an ocular tumor, the method comprising:
a) determining a first therapeutically effective dose of radiation for treating the ocular tumor;
b) administering a PKC inhibitor and a c-MET inhibitor to the patient as a neoadjuvant therapy over a period of time sufficient to reduce the size of the ocular tumor; and
c) irradiating the reduced-size ocular tumor with a second therapeutically effective dose of radiation at least 10% less than the first therapeutically effective dose of radiation.

In an aspect, provided herein is a method of treating a patient having uveal melanoma with an ocular tumor of a size indicated for an interventional procedure comprising irradiating the ocular tumor, the method comprising:
a) administering a PKC inhibitor and a c-MET inhibitor to the patient as neoadjuvant therapy;
b) reducing the size of the ocular tumor by at least 10%; and
c) irradiating the reduced-size ocular tumor with a therapeutically effective dose of radiation, wherein the therapeutically effective dose of radiation is less than a dose of radiation required to irradiate the ocular tumor in the absence of neoadjuvant therapy.

In an embodiment, the step (b) is reducing the size of the ocular tumor to dimensions of 5-19 mm LBD and less than 2.5 mm apical height.

In an aspect, provided herein is a method of treating uveal melanoma in a patient having an ocular tumor with a size defined by a set of dimensions comprising (i) greater than 19 mm LBD and any apical height, (ii) any LBD and greater than 10 mm apical height, (iii) greater than 8 mm apical height, any LBD, and the tumor is located close to the optic nerve, or (iv) less than or equal to 19 mm LBD and 2.5-10 mm apical height, the method comprising
a) administering a PKC inhibitor and administering a c-MET inhibitor to the patient as a neoadjuvant therapy; and
b) reducing the size of the ocular tumor defined by a set of dimensions having 5-19 mm LBD and less than 2.5 mm apical height. In an embodiment, the method further comprising treating the reduced-size ocular tumor with an interventional procedure.

In an aspect, provided herein is a method of treating uveal melanoma in a patient having an ocular tumor with a size defined by a set of dimensions comprising (i) greater than 19 mm LBD and any apical height, (ii) any LBD and greater than 10 mm apical height, or (iii) greater than 8 mm apical height, any LBD, and the tumor is located close to the optic nerve, the method comprising
a) administering a PKC inhibitor and administering a c-MET inhibitor to the patient as a neoadjuvant therapy;
b) reducing the size of the ocular tumor to dimensions of less than or equal to 19 mm LBD and 2.5-10 mm apical height; and
c) treating the reduced-size ocular tumor with an interventional procedure.

In another aspect, provided herein is a method of treating a patient having uveal melanoma with an ocular tumor with a size indicated for an interventional procedure comprising enucleation of the eye, the method comprising
a) administering a PKC inhibitor and administering a c-MET inhibitor to the patient as a neoadjuvant therapy;
b) reducing the size of the ocular tumor by at least 10%; and
c) treating the reduced-size ocular tumor with an interventional procedure other than enucleation.

In another aspect, provided herein is a method of treating a patient having uveal melanoma with an ocular tumor with a size indicated for an interventional procedure comprising enucleation of the eye, the method comprising
a) administering a PKC inhibitor and administering a c-MET inhibitor to the patient as a neoadjuvant therapy;
b) reducing the size of the ocular tumor by at least 10%; and
c) treating the reduced-size ocular tumor with an interventional procedure other than enucleation.

In an embodiment, the interventional procedure comprises local surgical resection of the tumor, irradiating the tumor, or any combination thereof. In another embodiment, the interventional procedure is local surgical resection of the tumor. In yet another embodiment, the interventional procedure is irradiating the tumor.

In some embodiments, the PKC inhibitor is a compound of Formula I:

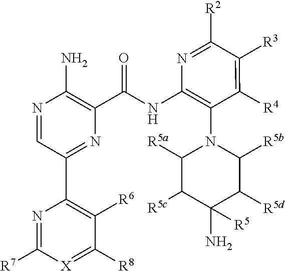

or a pharmaceutically acceptable salt thereof,
wherein:
X is N or CR;
R, R², R³ and R⁴ are each independently H, ²H, halo, OH, C₁₋₃ alkoxy, C₁₋₃ haloalkyl or C₁₋₃ alkyl, said C₁₋₃ alkyl optionally substituted with OH, C₁₋₃ alkoxy or C₁₋₃ haloalkoxy;
R⁵ is independently H, ²H, or C₁₋₃ alkyl, said C₁₋₃ alkyl optionally substituted with F, OH, C₁₋₃ alkoxy, or C₁₋₃ haloalkoxy;
R^{5a} and R^{5b} are each independently H, ²H, or C₁₋₃ alkyl, said C₁₋₃ alkyl optionally substituted with F, OH or C₁₋₃ alkoxy, or R^{5a} and R^{5b} are joined together forming a methylene or ethylene bridging group;
R^{5c} and R^{5d} are each independently H, ²H, F, OH, C₁₋₃ alkoxy, or C₁₋₃ alkyl, said C₁₋₃ alkyl optionally substituted with F, OH or C₁₋₃ alkoxy, or R^{5c} and R^{5d} are joined together forming a methylene, ethylene or —CH₂—O— bridging group; and
R⁶, R⁷ and R⁸ are each independently selected from H, ²H, halo, C₁₋₃ alkyl, C₁₋₃ haloalkyl, C₁₋₃ alkoxy, C₁₋₃ haloalkoxy, C₃-7 cycloalkyl and 4-7 membered heterocycloalkyl having 1 to 3 heteroatoms selected from N, O and S, said C₁₋₃ alkyl optionally substituted with F, OH, C₁₋₃ alkoxy or C₁₋₃ haloalkoxy.

In another embodiment, the PKC inhibitor is Compound 1:

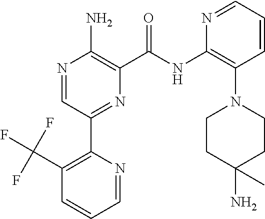 [SMILES: CC1(N)CCN(c2cccnc2NC(=O)c2nc(-c3ncccc3C(F)(F)F)cnc2N)CC1]

or a pharmaceutically acceptable salt thereof. In another embodiment, the PKC inhibitor is Compound 1.

In yet another embodiment, the c-MET inhibitor is a compound of Formula II:

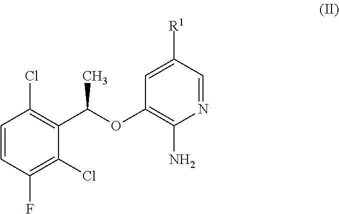

or a pharmaceutically acceptable salt thereof,
wherein:
R¹ is a furan, thiopene, pyrrole, pyrroline, pyrrolidine, dioxolane, oxazole, thiazole, imidazole, imidazoline, imidazolidine, pyrazole, pyrazoline, pyrazolidine, isoxazole, isothiazole, oxadiazole, triazole, thiadiazole, pyran, pyridine, piperidine, dioxane, morpholine, dithiane, thiomorpholine, pyridazine, pyrimidine, pyrazine, piperazine, triazine, trithiane, azitidine or phenyl; each of which is optionally substituted by R³;
each R³ is independently halogen, C₁₋₁₂ alkyl, C₂₋₁₂ alkenyl, C₂₋₁₂ alkynyl, C₃₋₁₂ cycloalkyl, C₆₋₁₂ aryl, 3-12 membered heterocycloalkyl, or 5-12 membered heteroaryl.

In still another embodiment, the c-MET inhibitor is Compound 2:

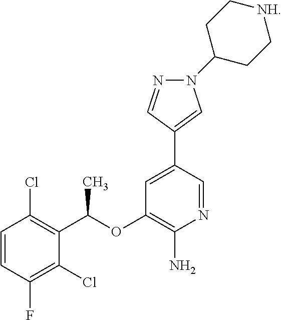 [SMILES: C[C@@H](Oc1cc(-c2cnn(C3CCNCC3)c2)cnc1N)c1c(Cl)ccc(F)c1Cl]

or a pharmaceutically acceptable salt thereof. In another embodiment, the c-MET inhibitor is Compound 2.

In another embodiment, the c-MET inhibitor can be a small molecule inhibitor of c-MET. The c-MET inhibitor can be an inhibitor of intracellular ATP-cMET ligand-receptor binding. Examples of the c-MET inhibitor include crizotinib, capmatinib, cabozantinib, tivantinib, or a combination thereof. In another embodiment, the c-MET Inhibitor is selected from the group consisting of crizotinib, capmatinib, cabozantinib, tivantinib, or a combination thereof.

In another embodiment, the method further comprises continuing administering a PKC inhibitor and administering a c-MET inhibitor to the patient as an adjuvant therapy during the period of the interventional procedure.

In yet another embodiment, the method further comprises continuing administering a PKC inhibitor and administering a c-MET inhibitor to the patient as an adjuvant therapy after the completion of the interventional procedure.

In still another embodiment, the PKC inhibitor and the c-MET inhibitor are administered orally.

In an embodiment, the method further comprises preserving the eye globe which had the ocular tumor.

In another embodiment, the method further comprises preserving a function of an eye which had the ocular tumor. In yet another embodiment, preserving the function of the eye which had the ocular tumor comprises at least partially preserving the eye function.

In still another embodiment, the method further comprises preventing irreversible vision loss. In an embodiment, the method further comprises preserving visual symptoms. In another embodiment, the method further comprises preserving depth perception. In yet another embodiment, the method further comprises preserving vision for greater than at least 1 year. In still another embodiment, the method further comprises preventing metastasis. In an embodiment, the method further comprises delaying onset of metastasis.

In another embodiment, the ocular tumor is spatially separated from the optic nerve.

In yet another embodiment, the ocular tumor, prior to administering the neoadjuvant therapy, has a size from 5 mm to 19 mm LBD and less than 2.5 mm apical height. In still another embodiment, the ocular tumor, prior to administering the neoadjuvant therapy, has a size less than or equal to 19 mm LBD and 2.5-10 mm apical height.

In another embodiment, the ocular tumor, prior to administering the neoadjuvant therapy, has a size greater than 19 mm LBD. In yet another embodiment, the ocular tumor, prior to administering the neoadjuvant therapy, has a size greater than 10 mm apical height and any size LBD. In still another embodiment, the ocular tumor, prior to administering the neoadjuvant therapy, has a size greater than 8 mm apical height and the tumor is located close to the optic nerve.

In an embodiment, the ocular tumor, prior to administering the neoadjuvant therapy, has a size of greater than 6 mm and less than 12 mm apical height, and not greater than 16 mm LBD. In another embodiment, the ocular tumor, prior to administering the neoadjuvant therapy, has a size of greater than 3 mm and less than 8 mm apical height, and not greater than 16 mm LBD. In yet another embodiment, the ocular tumor, prior to administering the neoadjuvant therapy, has a size of greater than 8 mm apical height, and greater than 16 mm LBD. In still another embodiment, the ocular tumor, prior to administering the neoadjuvant therapy, is not greater than 16 mm LBD. In an embodiment, the size of the ocular tumor, prior to administering the neoadjuvant therapy, is greater than 10 mm in apical height.

In another embodiment, the size of the ocular tumor after administering the neoadjuvant therapy but prior to the interventional procedure is reduced by at least 20%. In another embodiment, the size of the ocular tumor after administering the neoadjuvant therapy but prior to the interventional procedure is reduced by at least 30%. In another embodiment, the size of the ocular tumor after administering the neoadjuvant therapy but prior to the interventional procedure is reduced by at least 40%. In yet another embodiment, the size of the ocular tumor after administering the neoadjuvant therapy but prior to the interventional procedure is reduced by at least 50%. In another embodiment, the size of the ocular tumor after administering the neoadjuvant therapy but prior to the interventional procedure is reduced by at least 10%, at least 15%, at least 20%, at least 25%, at least 30%, at least 35%, at least 40%, at least 45%, at least 50%, at least 60%, at least 65%, at least 70%, or at least 75%.

In another embodiment, the LBD of the ocular tumor after administering the neoadjuvant therapy but prior to the interventional procedure is reduced by at least 1 mm, at least 2 mm, at least 3 mm, at least 4 mm, at least 5 mm, at least 6 mm, at least 7 mm, at least 8 mm, at least 9 mm, at least 10 mm, at least 11 mm, at least 12 mm, at least 13 mm, at least 14 mm, or at least 15 mm. In another embodiment, the LBD of the ocular tumor after administering the neoadjuvant therapy but prior to the interventional procedure is reduced by at least 1 mm, at least 2 mm, at least 3 mm, at least 4 mm, or at least 5 mm.

In another embodiment, the apical height of the ocular tumor after administering the neoadjuvant therapy but prior to the interventional procedure is reduced by at least 1 mm, at least 2 mm, at least 3 mm, at least 4 mm, at least 5 mm, at least 6 mm, at least 7 mm, at least 8 mm, at least 9 mm, at least 10 mm, at least 11 mm, at least 12 mm, at least 13 mm, at least 14 mm, or at least 15 mm. In another embodiment, the apical height LBD of the ocular tumor after administering the neoadjuvant therapy but prior to the interventional procedure is reduced by at least 1 mm, at least 2 mm, at least 3 mm, at least 4 mm, or at least 5 mm.

In still another embodiment, the PKC inhibitor, or an equivalent dose of a pharmaceutically acceptable salt thereof, is administered at a dose of about 200 mg to 400 mg two times per day (BID). In another embodiment, the PKC inhibitor, or an equivalent dose of a pharmaceutically acceptable salt thereof, is administered at a dose of about 300 mg two times per day (BID). In an embodiment, the c-MET inhibitor, or an equivalent dose of a pharmaceutically acceptable salt thereof, is administered at a dose of about 200 mg BID. In another embodiment, the PKC inhibitor, or an equivalent dose of a pharmaceutically acceptable salt thereof, is administered at a dose of about 400 mg to about 600 mg daily; and the c-MET inhibitor, or an equivalent dose of a pharmaceutically acceptable salt thereof, is administered at a dose of about 400 mg to about 500 mg daily.

In yet another embodiment, the PKC inhibitor, or an equivalent dose of a pharmaceutically acceptable salt thereof and the c-MET inhibitor, or an equivalent dose of a pharmaceutically acceptable salt thereof, are both administered daily for at least 7 days, at least 14 days, at least 21, days, at least 28 days, at least 56 days, at least 84 days, at least, 112 days, at last 140 days, or at least 168 days.

In still another embodiment, the interventional procedure is selected from the group consisting of plaque brachytherapy (PBT), external beam radiotherapy (EBRT), stereotactic radiosurgery (SRS), and any combination thereof. In an embodiment, the interventional procedure is a globe preserving treatment (GPT). In still another embodiment, the interventional procedure is plaque brachytherapy (PBT). In still another embodiment, the interventional procedure is external beam radiotherapy (EBRT). In still another embodiment, the interventional procedure is stereotactic radiosurgery (SRS). In still another embodiment, the plaque brachytherapy (PBT) is Ruthenium plaque brachytherapy.

In another embodiment, the interventional procedure is a globe preserving treatment (GPT). In yet another embodiment, the globe preserving treatment is selected from the group consisting of limited surgical resection or ablation (LSRA), radiation therapy (RT), brachytherapy (BT), external beam radiotherapy (EBRT), stereotactic radiosurgery (SRS), radiation therapy alone (RTA), radiation therapy and supplemental laser therapy (RT+SLT), and any combination thereof.

Exemplary interventional procedures can be found, at least, in (i) Foti P V et al., Diagnostic methods and therapeutic options of uveal melanoma with emphasis on MR imaging-Part II: treatment indications and complications. Insights Imaging. 2021 Jun. 4; 12 (1): 67, and (ii) Rao Y J, et al., Patterns of care and survival outcomes after treatment for uveal melanoma in the post-coms era (2004-2013): a surveillance, epidemiology, and end results analysis. J Contemp Brachytherapy. 2017 October; 9 (5): 453-465, the contents of which are incorporated in their entirety.

In still another embodiment, the uveal melanoma is a solid tumor harboring GNAQ or GNA11 mutations. In an embodiment, the patient has additional non-ocular tumor. In an embodiment, the non-ocular tumor is metastatic uveal melanoma (MUM). In an embodiment, the ocular tumor is malignant. In another embodiment, the ocular tumor is not malignant. In another embodiment, the ocular tumor is intraocular tumor.

In another embodiment, Compound 1, or a pharmaceutically acceptable salt thereof, is administered at a dose of about 400 mg to about 600 mg daily of free base equivalent of Compound 1; and Compound 2, or a pharmaceutically acceptable salt thereof, is administered at a dose of about 400 mg to about 500 mg daily of free base equivalent of Compound 2. In another embodiment, Compound 1, or a pharmaceutically acceptable salt thereof, is administered at a dose of about 200 mg to about 600 mg daily of free base equivalent of Compound 1; and Compound 2, or a pharmaceutically acceptable salt thereof, is administered at a dose of about 400 mg to about 500 mg daily of free base equivalent of Compound 2.

In an embodiment, the uveal melanoma is metastatic uveal melanoma. In another embodiment, the uveal melanoma is a solid tumor harboring GNAQ or GNA11 mutations. In another embodiment, the uveal melanoma is metastatic uveal melanoma harboring GNAQ or GNA11 mutations.

In another embodiment, Compound 1, or a pharmaceutically acceptable salt thereof, is administered at a dose of about 100 mg once daily (QD) of free base equivalent of Compound 1. In another embodiment, Compound 1, or a pharmaceutically acceptable salt thereof, is administered at a dose of about 200 mg once daily (QD) of free base equivalent of Compound 1. In yet another embodiment, Compound 1, or a pharmaceutically acceptable salt thereof, is administered at a dose of about 300 mg once daily (QD) of free base equivalent of Compound 1. In yet another embodiment, Compound 1, or a pharmaceutically acceptable salt thereof, is administered at a dose of about 400 mg once daily (QD) of free base equivalent of Compound 1.

In another embodiment, Compound 1, or a pharmaceutically acceptable salt thereof, is administered at a dose of about 100 mg BID of free base equivalent of Compound 1. In yet another embodiment, Compound 1, or a pharmaceutically acceptable salt thereof, is administered at a dose of about 200 mg BID of free base equivalent of Compound 1. In still another embodiment, Compound 1, or a pharmaceutically acceptable salt thereof, is administered at a dose of about 300 mg BID of free base equivalent of Compound 1. In another embodiment, Compound 1, or a pharmaceutically acceptable salt thereof, is administered at a dose of about 400 mg BID of free base equivalent of Compound 1.

In another embodiment, Compound 2, or a pharmaceutically acceptable salt thereof, is administered at a dose of about 200 mg BID of free base equivalent of Compound 2. In an embodiment, Compound 2, or a pharmaceutically acceptable salt thereof, is administered at a dose of about 250 mg BID of free base equivalent of Compound 2. In another embodiment, Compound 2, or a pharmaceutically acceptable salt thereof, is administered at a dose of about 200 mg once daily (QD) of free base equivalent of Compound 2. In yet another embodiment, Compound 2, or a pharmaceutically acceptable salt thereof, is administered at a dose of about 250 mg once daily (QD) of free base equivalent of Compound 2.

In another embodiment, the administration of Compound 1, or a pharmaceutically acceptable salt thereof, is uninterrupted during a dosing schedule comprising at least one 7-day dosing cycle.

In yet another embodiment, Compound 1, or a pharmaceutically acceptable salt thereof, and Compound 2, or a pharmaceutically acceptable salt thereof, are administered in a single formulation. In still another embodiment, Compound 1, or a pharmaceutically acceptable salt thereof, and Compound 2, or a pharmaceutically acceptable salt thereof, are administered in a single formulation further comprising one or more pharmaceutically acceptable carriers.

In an embodiment, Compound 1, or a pharmaceutically acceptable salt thereof, and Compound 2, or a pharmaceutically acceptable salt thereof, are administered separately.

In another embodiment, the treatment comprises administering Compound 1, or a pharmaceutically acceptable salt thereof, and Compound 2, or a pharmaceutically acceptable salt thereof, at substantially the same time. In yet another embodiment, the treatment comprises administering Compound 1, or a pharmaceutically acceptable salt thereof, and Compound 2, or a pharmaceutically acceptable salt thereof, at different times.

In still another embodiment, Compound 1, or a pharmaceutically acceptable salt thereof, is administered to the subject, followed by administration of Compound 2, or a pharmaceutically acceptable salt thereof. In an embodiment, Compound 2, or a pharmaceutically acceptable salt thereof, is administered to the subject, followed by administration of Compound 1, or a pharmaceutically acceptable salt thereof.

In an embodiment, administering Compound 1, or a pharmaceutically acceptable salt thereof, and Compound 2, or a pharmaceutically acceptable salt thereof, reduces the size of one or more lesions of the uveal melanoma or the solid tumor harboring GNAQ or GNA11 mutations in said subject.

In another embodiment, administering Compound 1, or a pharmaceutically acceptable salt thereof, and Compound 2, or a pharmaceutically acceptable salt thereof, decreases the growth rate of one of more lesions of the uveal melanoma or the solid tumor harboring GNAQ or GNA11 mutations in said subject.

In another embodiment, the method of treating uveal melanoma in a subject in need thereof can further comprise: (i) following administration of Compound 1, or a pharmaceutically acceptable salt thereof, and Compound 2, or a pharmaceutically acceptable salt thereof, to the patient in need thereof according to the dosing regimen of any of the aforementioned embodiments over a treatment period comprising at least 4 consecutive 7-day dosing cycles, (ii) increasing the dose of Compound 1, or a pharmaceutically acceptable salt thereof, to 300 mg BID or 600 mg total daily as applicable, and/or (iii) lowering the dose of Compound 2, or a pharmaceutically acceptable salt thereof, to 200 mg BID or 250 mg total daily as applicable.

In yet another embodiment, the method of treating uveal melanoma in a subject in need thereof can further comprise: continuing to administer Compound 1, or a pharmaceutically acceptable salt thereof, and Compound 2, or a pharmaceutically acceptable salt thereof, uninterrupted during a dosing schedule comprising at least two or more consecutive 7-day dosing cycles, or preferably at least four or more consecutive 7-day dosing cycles. In particular embodiments, Compound 1, or a pharmaceutically acceptable salt thereof, and Compound 2, or a pharmaceutically acceptable salt thereof, are continued to be co-administered uninterrupted during a dosing schedule comprising at least eight or more, twelve or more, twenty-four or more, forty-eight or more or ninety-six or more consecutive 7-day dosing cycles. In an embodiment, Compound 1, or a pharmaceutically acceptable salt thereof, is continued to be co-administered uninterrupted during a dosing schedule comprising at least eight or more, twelve or more, twenty-four or more, forty-eight or more or ninety-six or more consecutive 7-day dosing cycles. In another embodiment, Compound 2, or a pharmaceutically acceptable salt thereof, is continued to be co-administered uninterrupted during a dosing schedule comprising at least eight or more, twelve or more, twenty-four or more, forty-eight or more or ninety-six or more consecutive 7-day dosing cycles.

In yet another embodiment, the therapeutically effective dose of radiation is determined based on the ocular tumor size, location, or a combination thereof.

In still another embodiment, the size and location of the ocular tumor is determined by CT scan, MRI, or a combination thereof. In another embodiment, the size or location of the ocular tumor is determined by CT scan, MRI, or a combination thereof. In another embodiment, the size and/or location of the ocular tumor is determined by ultrasound.

In an embodiment, the therapeutically effective dose of radiation is determined using Plaque Simulator. In another embodiment, the Plaque Simulator is version 6 (PS6).

In yet another embodiment, the method further comprises continuing administering a PKC inhibitor and a c-MET inhibitor to the patient as an adjuvant therapy during the period of the irradiation. In still another embodiment, the method further comprises continuing administering a PKC inhibitor and a c-MET inhibitor to the patient as an adjuvant therapy after the completion of the irradiation.

In an embodiment, determining a first therapeutically effective dose of radiation for treating the ocular tumor comprises determining a therapeutically effective dose of radiation to fovea before administering the PKC inhibitor and a c-MET inhibitor to the patient. In yet another embodiment, irradiating the reduced-size ocular tumor with a second therapeutically effective dose of radiation comprises determining a therapeutically effective dose of radiation to fovea. In still another embodiment, irradiating the reduced-size ocular tumor with a therapeutically effective dose of radiation comprises determining a therapeutically effective dose of radiation to fovea.

Exemplary lengths of time associated with the course of the treatment methods is about five years, about 4 years, about 3 years, about 2 years, about 1 year, about 11 months, about 10 months, about 9 months, about 8 months, about 7 months, about 6 months, about 5 months, about 4 months, about 3 months, about 2 months, or about 1 month.

Exemplary lengths of time associated with the course of the treatment methods is about five years and so on; or any days, weeks, months, or years in between; for example a treatment cycle can include 5 months and additional weeks and/or days, or one year and additional months, weeks, and/or days, and the like.

In some embodiments, Compound 1, or a pharmaceutically acceptable salt thereof, and Compound 2, or a pharmaceutically acceptable salt thereof, are administered continuously (i.e., a continuous treatment until termination).

The UM to be treated can include one or more of a number of mutations, including a substitution mutation, an insertion mutation, and/or a deletion in GNAQ or GNA11 mutation. In some aspects, the GNAQ or GNA11 mutation is a gain of function mutation. In some aspects, the GNAQ or GNA11 mutation activates the PKC signaling pathway. In various aspects, the GNAQ or GNA11 mutation can be the substitution of glutamine in codon 209 (Q209) and/or a substitution of arginine in codon 183 (R183). The GNAQ or GNA11 mutation can be a substitution other than glutamine in codon 209 (Q209), other than a substitution of arginine in codon 183 (R183), or other than both. In some aspects, the GNAQ mutation is one of Q209P, Q209L, Q209H, Q209K, or Q209Y, or the GNA11 mutation is one of Q209P, Q209L, Q209K or Q209H. In further aspects, the GNAQ mutation can be R183Q, or the GNA11 mutation can be R183C or R183H. In yet further examples, the GNAQ or GNA11 mutation is at one or more of R256, L279, R166, A168, R210, R213, R166, A231, A342, D333, G171, R147, R73, T47, E191, E221, R149, T175, T379, T85, A86, E163, D195, E319, E191, E280, E49, P293, R300, R338, R60, D155, D205, D321, 1226, R37, or V240. In further examples, the UM can comprise one or more of a Q209P, Q209L, Q209H, Q209K, Q209Y, or R183Q mutation in GNAQ, or the UM can comprise one or more of a Q209P, Q209L, Q209H, or Q209K mutation in GNA11. Additional examples of mutations in GNAQ or GNA11 are described in WO 2020/146355, which is incorporated by reference herewith in its entirety.

Exemplary lengths of time associated with the course of the treatment methods disclosed herein include: about one week; about two weeks; about three weeks; about four weeks; about five weeks; about six weeks; about seven weeks; about eight weeks; about nine weeks; about ten weeks; about eleven weeks; about twelve weeks; about thirteen weeks; about fourteen weeks; about fifteen weeks; about sixteen weeks; about seventeen weeks; about eighteen weeks; about nineteen weeks; about twenty weeks; about twenty-one weeks; about twenty-two weeks; about twenty-three weeks; about twenty four weeks; about 4 months; about seven months; about eight months; about nine months; about ten months; about eleven months; about twelve months; about thirteen months; about fourteen months; about fifteen months; about sixteen months; about seventeen months; about eighteen months; about nineteen months; about twenty months; about twenty one months; about twenty-two months; about twenty-three months; about twenty-four months; about thirty months; about three years; about four years and about five years and so on; or any days, weeks, months, or years in between; for example a treatment cycle can include 5 months and additional weeks and/or days, or one year and additional months, weeks, and/or days, and the like.

In an embodiment of the methods, the method involves the administration of a therapeutically effective amount of a combination or composition comprising compounds provided herein, or pharmaceutically acceptable salts thereof, to a subject (including, but not limited to a human or animal) in need of treatment (including a subject identified as in need).

In another embodiment of the methods, the treatment includes co-administering the amount of Compound 1 or a pharmaceutically acceptable salt thereof and the amount of Compound 2 or a pharmaceutically acceptable salt thereof. In an embodiment, the amount of Compound 1 or a pharmaceutically acceptable salt thereof and the amount of Compound 2 or a pharmaceutically acceptable salt thereof are in a single formulation or unit dosage form. In still other embodiments, the amount of Compound 1 or a pharmaceutically acceptable salt thereof and the amount of Compound 2 or a pharmaceutically acceptable salt thereof are in a separate formulations or unit dosage forms.

In the foregoing methods, the treatment can include administering the amount of Compound 1 or a pharmaceutically acceptable salt thereof and the amount of Compound 2 or a pharmaceutically acceptable salt thereof at substantially the same time or administering the amount of Compound 1 or a pharmaceutically acceptable salt thereof and the amount of Compound 2 or a pharmaceutically acceptable salt thereof at different times. In some embodiments of the foregoing methods, the amount of Compound 1 or a pharmaceutically acceptable salt thereof and/or the amount of Compound 2 or a pharmaceutically acceptable salt thereof is administered at dosages that would not be effective when one or both of Compound 1 or a pharmaceutically acceptable salt thereof and Compound 2 or a pharmaceutically acceptable salt thereof is administered alone, but which amounts are effective in combination.

In one embodiment of the foregoing methods, the treatment reduces the size of one of more lesions of the uveal melanoma in said subject.

In one embodiment of the foregoing methods, the treatment reduces the size of one of more lesions of the metastatic uveal melanoma in said subject.

In one embodiment of the foregoing methods, the treatment decreases the growth rate of one of more lesions of the uveal melanoma in said subject.

In one embodiment of the foregoing methods, the treatment decreases the growth rate of one of more lesions of the metastatic uveal melanoma in said subject.

In another embodiment, the subject is a First-Line MUM subject.

### Administration/Dosage/Formulations

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

In particular, the selected dosage level will depend upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could begin administration of the pharmaceutical composition to dose the disclosed compound at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of the disclosed compound calculated to produce the desired therapeutic effect in association with the required pharmaceutical vehicle. The dosage unit forms are dictated by and directly dependent on (a) the unique characteristics of the disclosed compound and the particular therapeutic effect to be achieved, and (b) the limitations inherent in the art of compounding/formulating such a disclosed compound for the treatment of pain, a depressive disorder, or drug addiction in a patient.

In one embodiment, the compounds provided herein are formulated using one or more pharmaceutically acceptable excipients or carriers. In one embodiment, the pharmaceutical compositions provided herein comprise a therapeutically effective amount of a disclosed compound and a pharmaceutically acceptable carrier.

The optimum ratios, individual and combined dosages, and concentrations of the drug compounds that yield efficacy without toxicity are based on the kinetics of the active ingredients' availability to target sites, and are determined using methods known to those of skill in the art.

Routes of administration of any of the compositions discussed herein include oral, nasal, rectal, intravaginal, parenteral, buccal, sublingual or topical. The compounds may be formulated for administration by any suitable route, such as for oral or parenteral, for example, transdermal, transmucosal (e.g., sublingual, lingual, (trans) buccal, (trans) urethral, vaginal (e.g., trans- and perivaginally), (intra) nasal and (trans) rectal), intravesical, intrapulmonary, intraduodenal, intragastrical, intrathecal, subcutaneous, intramuscular, intradermal, intra-arterial, intravenous, intrabronchial, inhalation, and topical administration. In one embodiment, the preferred route of administration is oral.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions are not limited to the particular formulations and compositions that are described herein.

For oral application, particularly suitable are tablets, dragees, liquids, drops, suppositories, or capsules, caplets and gel caps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For parenteral administration, the disclosed compounds may be formulated for injection or infusion, for example, intravenous, intramuscular or subcutaneous injection or infusion, or for administration in a bolus dose or continuous infusion. Suspensions, solutions or emulsions in an oily or aqueous vehicle, optionally containing other formulatory agents such as suspending, stabilizing or dispersing agents may be used.

### Kits

In an aspect, the present disclosure provides a kit for treating uveal melanoma, comprising Compound 1, or an equivalent dose of a pharmaceutically acceptable salt thereof, in a unit dosage of about 400 mg to about 600 mg, and Compound 2, or an equivalent dose of a pharmaceutically acceptable salt thereof, in a unit dosage of about 400 mg to about 500 mg. In another embodiment, the present disclosure provides a kit for treating uveal melanoma, comprising Compound 1, or an equivalent dose of a pharmaceutically acceptable salt thereof, in an amount of about 300 mg, and Compound 2, or an equivalent dose of a pharmaceutically acceptable salt thereof, in an amount of about 200 mg. In another embodiment, the present disclosure provides a kit for treating uveal melanoma, comprising Compound 1, or an equivalent dose of a pharmaceutically acceptable salt thereof, in an amount of about 200 mg, and Compound 2, or an equivalent dose of a pharmaceutically acceptable salt thereof, in an amount of about 200 mg. In another embodiment, the present disclosure provides a kit for treating uveal melanoma, comprising Compound 1, or an equivalent dose of a pharmaceutically acceptable salt thereof, in an amount from about 400 mg per day to about 600 mg per day, and Compound 2, or an equivalent dose of a pharmaceutically acceptable salt thereof, in an amount from about 400 mg per day to about 500 mg per day. In some embodiments, the kit further comprises packaging and instructions. In yet another embodiment, the uveal melanoma is metastatic uveal melanoma. In yet another embodiment, the metastatic uveal melanoma is a solid tumor harboring GNAQ or GNA11 mutations.

In certain embodiments, the kit comprises a pharmaceutical product comprising a pharmaceutical composition comprising Compound 1, or a pharmaceutically acceptable salt thereof, and a pharmaceutically acceptable carrier or diluent; and a pharmaceutical composition comprising Compound 2, or a pharmaceutically acceptable salt thereof, and a pharmaceutically acceptable carrier or diluent.

In additional embodiments, pharmaceutical kits are provided. The kit includes a sealed container approved for the storage of pharmaceutical compositions, the container containing one of the above-described pharmaceutical compositions. In some embodiments, the sealed container minimizes the contact of air with the ingredients, e.g. an airless bottle. In other embodiments, the sealed container is a sealed tube. An instruction for the use of the composition and the information about the composition are to be included in the kit.

In a particular embodiment, the compounds of the combination can be dosed on the same schedule, whether by administering a single formulation or unit dosage form containing all of the compounds of the combination, or by administering separate formulations or unit dosage forms of the compounds of the combination. However, some of the compounds used in the combination may be administered more frequently than once per day, or with different frequencies that other compounds in the combination. Therefore, in one embodiment, the kit contains a formulation or unit dosage form containing all of the compounds in the combination of compounds, and an additional formulation or unit dosage form that includes one of the compounds in the combination of agents, with no additional active compound, in a container, with instructions for administering the dosage forms on a fixed schedule.

The kits provided herein comprise prescribing information, for example, to a patient or health care provider, or as a label in a packaged pharmaceutical formulation. Prescribing information may include for example efficacy, dosage and administration, contraindication and adverse reaction information pertaining to the pharmaceutical formulation.

In all of the foregoing the combination of compounds of the invention can be administered alone, as mixtures, or with additional active agents.

A kit provided herein can be designed for conditions necessary to properly maintain the components housed therein (e.g., refrigeration or freezing). A kit can contain a label or packaging insert including identifying information for the components therein and instructions for their use (e.g., dosing parameters, clinical pharmacology of the active ingredient(s), including mechanism(s) of action, pharmacokinetics and pharmacodynamics, adverse effects, contraindications, etc.).

Each component of the kit can be enclosed within an individual container, and all of the various containers can be within a single package. Labels or inserts can include manufacturer information such as lot numbers and expiration dates. The label or packaging insert can be, e.g., integrated into the physical structure housing the components, contained separately within the physical structure, or affixed to a component of the kit (e.g., an ampule, syringe or vial).

### Non-Limiting Exemplary Embodiments

In further embodiments 1 to 108 below, the present disclosure includes:
1. In Embodiment 1 is a method of treating uveal melanoma in a patient having an ocular tumor, the method comprising
   a) administering a PKC inhibitor and administering a c-MET inhibitor to the patient as a neoadjuvant therapy;
   b) reducing the size of the ocular tumor by at least 10%; and
   c) treating the reduced-size ocular tumor with an interventional procedure.
2. In Embodiment 2 is the method of embodiment 1, wherein the ocular tumor is defined by a set of dimensions comprising (i) greater than or equal to 5 mm largest basal diameter (LBD) and any apical height, or (ii) any LBD and greater than 3 mm apical height.
3. In Embodiment 3 is a method of treating a patient having uveal melanoma with an ocular tumor with a size indicated for an interventional procedure comprising enucleation of the eye, the method comprising
   a) administering a PKC inhibitor and administering a c-MET inhibitor to the patient as a neoadjuvant therapy;
   b) reducing the size of the ocular tumor by at least 10%; and
   c) treating the reduced-size ocular tumor with an interventional procedure other than enucleation.
4. In Embodiment 4 is a method of treating a patient having uveal melanoma with an ocular tumor with a size indicated for an interventional procedure comprising irradiating the ocular tumor, the method comprising
   a) administering a PKC inhibitor and administering a c-MET inhibitor to the patient as a neoadjuvant therapy;
   b) reducing the size of the ocular tumor by at least 10%; and
   c) treating the reduced-size ocular tumor with an interventional procedure other than enucleation.
5. In Embodiment 5 is a method of treating uveal melanoma in a patient having an ocular tumor with a size defined by a set of dimensions comprising (i) greater than 16 mm LBD and any apical height; (ii) any LBD and greater than 8 mm apical height; or (iii) less than 16 mm LBD and 3-8 mm apical height, the method comprising
   a) administering a PKC inhibitor and administering a c-MET inhibitor to the patient as a neoadjuvant therapy;
   b) reducing the size of the ocular tumor to dimensions of less than 5 mm LBD and less than 3 mm apical height; and
   c) treating the reduced-size ocular tumor with an interventional procedure.
6. In Embodiment 6 is a method of treating uveal melanoma in a patient having an ocular tumor with a size defined by a set of dimensions comprising (i) greater than 16 mm LBD and any apical height; (ii) any LBD and greater than 8 mm apical height; or (iii) less than 16 mm LBD and 3-8 mm apical height, the method comprising
   a) administering a PKC inhibitor and administering a c-MET inhibitor to the patient; and
   b) reducing the size of the ocular tumor defined by a set of dimensions having less than 5 mm LBD and less than 3 mm apical height.
7. In Embodiment 7, the method of embodiment 6, further comprising treating the reduced-size ocular tumor with an interventional procedure.
8. In Embodiment 8 is a method of treating uveal melanoma in a patient having an ocular tumor with a size defined by a set of dimensions comprising (i) greater than 16 mm LBD and any apical height, or (ii) any LBD and greater than 8 mm apical height, the method comprising
   a) administering a PKC inhibitor and administering a c-MET inhibitor to the patient as a neoadjuvant therapy;
   b) reducing the size of the ocular tumor to dimensions of less than 16 mm LBD and less than 8 mm apical height; and
   c) treating the reduced-size ocular tumor with an interventional procedure.
9. In Embodiment 9, the method of embodiment 8, wherein step (b) is reducing the size of the ocular tumor to dimensions of less than 16 mm LBD and 3-8 mm apical height.
10. In Embodiment 10 is a method of treating uveal melanoma in a patient having an ocular tumor with a size defined by a set of dimensions comprising (i) greater than 19 mm LBD and any apical height, (ii) any LBD and greater than 10 mm apical height, (iii) greater than 8 mm apical height, any LBD, and the tumor is located close to the optic nerve, or (iv) less than or equal to 19 mm LBD and 2.5-10 mm apical height, the method comprising
   a) administering a PKC inhibitor and administering a c-MET inhibitor to the patient as a neoadjuvant therapy;
   b) reducing the size of the ocular tumor defined by a set of dimensions having 5-19 mm LBD and less than 2.5 mm apical height; and
   c) treating the reduced-size ocular tumor with an interventional procedure.
11. In Embodiment 11, the method of embodiment 10, wherein step (b) is reducing the size of the ocular tumor to dimensions of 5-19 mm LBD and less than 2.5 mm apical height.
12. In Embodiment 12 is a method of treating uveal melanoma in a patient having an ocular tumor with a size defined by a set of dimensions comprising (i) greater than 19 mm LBD and any apical height, (ii) any LBD and greater than 10 mm apical height, (iii) greater than 8 mm apical height, any LBD, and the tumor is located close to the optic nerve, or (iv) less than or equal to 19 mm LBD and 2.5-10 mm apical height, the method comprising
   a) administering a PKC inhibitor and administering a c-MET inhibitor to the patient as a neoadjuvant therapy; and
   b) reducing the size of the ocular tumor defined by a set of dimensions having 5-19 mm LBD and less than 2.5 mm apical height.
13. In Embodiment 13 is a method of treating uveal melanoma in a patient having an ocular tumor with a size defined by a set of dimensions comprising (i) up to 22 mm in LBD and (ii) up to 15 mm in apical height, the method comprising
   a) administering a PKC inhibitor and administering a c-MET inhibitor to the patient as a neoadjuvant therapy; and
   b) reducing the size of the ocular tumor by at least 10%.
14. In Embodiment 14 is a method of treating uveal melanoma in a patient having an ocular tumor with a size defined by a set of dimensions comprising (i) up to 22 mm in LBD or (ii) up to 15 mm in apical height, the method comprising
   a) administering a PKC inhibitor and administering a c-MET inhibitor to the patient as a neoadjuvant therapy; and
   b) reducing the size of the ocular tumor by at least 10%.
15. In Embodiment 15, the method of embodiment 13 or 14, wherein the patient had been recommended enucleation before the neoadjuvant therapy was administered.
16. In Embodiment 16 is a method of treating uveal melanoma in a patient having an ocular tumor with a size defined by a set of dimensions comprising (i) at least 6 mm in LBD and (ii) at least 3 mm in apical height, the method comprising
   a) administering a PKC inhibitor and administering a c-MET inhibitor to the patient as a neoadjuvant therapy; and
   b) reducing the size of the ocular tumor by at least 10%.
17. In Embodiment 17 is a method of treating uveal melanoma in a patient having an ocular tumor with a size defined by a set of dimensions comprising (i) at least 6 mm in LBD or (ii) at least 3 mm in apical height, the method comprising
   a) administering a PKC inhibitor and administering a c-MET inhibitor to the patient as a neoadjuvant therapy; and
   b) reducing the size of the ocular tumor by at least 10%.
18. In Embodiment 18, the method of embodiment 17, wherein the patients having sub-foveal or greater than 180-degree optic nerve involved tumors were excluded.
19. In Embodiment 19, the method of any one of embodiments 16 to 18, wherein the patient had been recommended irradiating the tumor before the neoadjuvant therapy was administered.
20. In Embodiment 20, method of any one of embodiments 12 to 19, further comprising treating the reduced-size ocular tumor with an interventional procedure.
21. In Embodiment 21 is a method of treating uveal melanoma in a patient having an ocular tumor with a size defined by a set of dimensions comprising (i) greater than 19 mm LBD and any apical height, (ii) any LBD and greater than 10 mm apical height, or (iii) greater than 8 mm apical height, any LBD, and the tumor is located close to the optic nerve, the method comprising
   a) administering a PKC inhibitor and administering a c-MET inhibitor to the patient as a neoadjuvant therapy;
   b) reducing the size of the ocular tumor to dimensions of less than or equal to 19 mm LBD and 2.5-10 mm apical height; and
   c) treating the reduced-size ocular tumor with an interventional procedure.
22. In Embodiment 22, the method of any one of embodiments 1 to 21, wherein the interventional procedure comprises local surgical resection of the tumor, irradiating the tumor, or any combination thereof.
23. In Embodiment 23, the method of any one of embodiments 1 to 22, the primary interventional procedure comprises local surgical resection of the tumor.
24. In Embodiment 24, the method of any one of embodiments 1 to 23, wherein the interventional procedure comprises irradiating the tumor.
25. In Embodiment 25, the method of any one of embodiments 1 to 21, wherein the interventional procedure is irradiating the tumor.
26. In Embodiment 26, is a method of treating uveal melanoma in a patient having an ocular tumor, the method comprising:
   a) determining a first therapeutically effective dose of radiation for treating the ocular tumor;
   b) administering a PKC inhibitor and administering a c-MET inhibitor to the patient as a neoadjuvant therapy;
   c) reducing the size of the ocular tumor by at least 10%; and
   d) irradiating the reduced-size ocular tumor with a second therapeutically effective dose of radiation less than the first therapeutically effective dose of radiation.
27. In Embodiment 27, is a method of treating uveal melanoma in a patient having an ocular tumor, the method comprising:
   a) determining a first therapeutically effective dose of radiation for treating the ocular tumor;
   b) administering a PKC inhibitor and administering a c-MET inhibitor to the patient as a neoadjuvant therapy;
   c) reducing the size of the ocular tumor; and
   d) irradiating the reduced-size ocular tumor with a second therapeutically effective dose of radiation at least 10% less than the first therapeutically effective dose of radiation.
28. In Embodiment 28, is a method of treating uveal melanoma in a patient having an ocular tumor, the method comprising:
   a) determining a first therapeutically effective dose of radiation for treating the ocular tumor;
   b) administering a PKC inhibitor and administering a c-MET inhibitor to the patient as a neoadjuvant therapy to reduce the size of the ocular tumor by at least 10%; and
   c) irradiating the reduced-size ocular tumor with a second therapeutically effective dose of radiation less than the first therapeutically effective dose of radiation.
29. In Embodiment 29, the method of embodiment 28 wherein the second therapeutically effective dose of radiation is less than the first therapeutically effective dose of radiation by at least 5%, 10%, 15%, 20%, 25%, 30%, 35%, 40%, 45%, 50%, 55%, 60%, 65&, 70%, 75%, 80%, 85%, or 90%.
30. In Embodiment 30, is a method of treating uveal melanoma in a patient having an ocular tumor, the method comprising:
   a) determining a first therapeutically effective dose of radiation for treating the ocular tumor;
   b) administering a PKC inhibitor and administering a c-MET inhibitor to the patient as a neoadjuvant therapy to reduce the size of the ocular tumor; and
   c) irradiating the reduced-size ocular tumor with a second therapeutically effective dose of radiation at least 10% less than the first therapeutically effective dose of radiation.
31. In Embodiment 31, is a method of treating uveal melanoma in a patient having an ocular tumor, the method comprising:
   a) determining a first therapeutically effective dose of radiation for treating the ocular tumor;
   b) administering a PKC inhibitor and administering a c-MET inhibitor to the patient as a neoadjuvant therapy over a period of time sufficient to reduce the size of the ocular tumor by at least 10%; and
   c) irradiating the reduced-size ocular tumor with a second therapeutically effective dose of radiation less than the first therapeutically effective dose of radiation.
32. In Embodiment 32, the method of embodiment 31 wherein the second therapeutically effective dose of radiation is less than the first therapeutically effective dose of radiation by at least 5%, 10%, 15%, 20%, 25%, 30%, 35%, 40%, 45%, 50%, 55%, 60%, 65&, 70%, 75%, 80%, 85%, or 90%.
33. In Embodiment 33, is a method of treating uveal melanoma in a patient having an ocular tumor, the method comprising:
   a) determining a first therapeutically effective dose of radiation for treating the ocular tumor;
   b) administering a PKC inhibitor and administering a c-MET inhibitor to the patient as a neoadjuvant therapy over a period of time sufficient to reduce the size of the ocular tumor; and
   c) irradiating the reduced-size ocular tumor with a second therapeutically effective dose of radiation at least 10% less than the first therapeutically effective dose of radiation.
34. In Embodiment 34, is a method of treating a patient having uveal melanoma with an ocular tumor of a size indicated for an interventional procedure comprising irradiating the ocular tumor, the method comprising:
   a) administering a PKC inhibitor and administering a c-MET inhibitor to the patient as neoadjuvant therapy;
   b) reducing the size of the ocular tumor by at least 10%; and
   c) irradiating the reduced-size ocular tumor with a therapeutically effective dose of radiation, wherein the therapeutically effective dose of radiation is less than a dose of radiation required to irradiate the ocular tumor in the absence of neoadjuvant therapy.
35. In Embodiment 35, the method of any one of embodiments 1 to 34, wherein the PKC inhibitor is Compound 1:

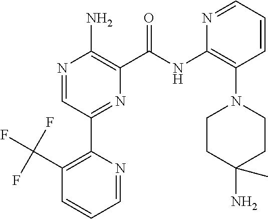 [SMILES: CC1(N)CCN(c2cccnc2NC(=O)c2nc(-c3ncccc3C(F)(F)F)cnc2N)CC1]

or a pharmaceutically acceptable salt thereof.
36. In Embodiment 36, the method of any one of embodiments 1 to 35, wherein the PKC inhibitor is Compound 1.
37. In Embodiment 37, the method of any one of embodiments 1 to 36, wherein the c-MET inhibitor is Compound 2:

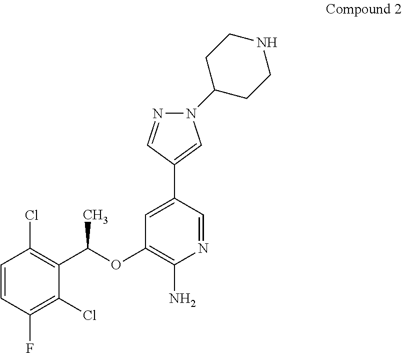 [SMILES: C[C@@H](Oc1cc(-c2cnn(C3CCNCC3)c2)cnc1N)c1c(Cl)ccc(F)c1Cl]

or a pharmaceutically acceptable salt thereof.
38. In Embodiment 38, the method of any one of embodiments 1 to 37, wherein the c-MET inhibitor is Compound 2.
39. In Embodiment 39, the method of any one of embodiments 1 to 38, wherein the interventional procedure is a globe preserving treatment (GPT).
40. In Embodiment 40, the method of any one of embodiments 1 to 39, wherein the interventional procedure is selected from the group consisting of plaque brachytherapy (PBT), external beam radiotherapy (EBRT), stereotactic radiosurgery (SRS), LASER, additional pharmacological agent therapy, and any combination thereof.
41. In Embodiment 41, the method of any one of embodiments 1 to 39, wherein the globe preserving treatment is selected from the group consisting of limited surgical resection or ablation (LSRA), radiation therapy (RT), brachytherapy (BT), external beam radiotherapy (EBRT), stereotactic radiosurgery (SRS), radiation therapy alone (RTA), radiation therapy and supplemental laser therapy (RT+SLT), and any combination thereof.
42. In Embodiment 42, the method of any one of embodiments 1 to 39, wherein the interventional procedure is selected from the group consisting of proton beam radiotherapy, transpupillary thermotherapy (TTT), thermotherapy, laser photocoagulation therapy, limited surgical resection or ablation (LSRA), radiation therapy (RT), brachytherapy (BT), external beam radiotherapy (EBRT), stereotactic radiosurgery (SRS), radiation therapy alone (RTA), radiation therapy and supplemental laser therapy (RT+SLT), charged-particles beam radiotherapy, and any combination thereof.
43. In Embodiment 43, the method of any one of embodiments 1 to 39, wherein the interventional procedure is brachytherapy (BT).
44. In Embodiment 44, the method of any one of embodiments 40 to 42, wherein stereotactic radiotherapy (SRS) is selected from the group consisting of gamma-knife, cyber knife and linear accelerator (LINAC),
45. In Embodiment 45, the method of any one of embodiments 1 to 44, wherein the method further comprises continuing administering a PKC inhibitor and administering a c-MET inhibitor to the patient as an adjuvant therapy during the period of the interventional procedure.
46. In Embodiment 46, the method of any one of embodiments 1 to 44, wherein the method further comprises continuing administering a PKC inhibitor and administering a c-MET inhibitor to the patient as an adjuvant therapy after the completion of the interventional procedure.
47. In Embodiment 47, the method of embodiment 46, wherein the adjuvant therapy is administered at least 1 week, at least 2 weeks, at least 3 weeks, at least 4 weeks, at least 5 weeks, or at least 6 weeks after the completion of the interventional procedure.
48. In Embodiment 48, the method of embodiment 46 or 47, wherein the adjuvant therapy is administered at least 4 weeks, at least 5 weeks, or at least 6 weeks after the completion of the interventional procedure.
49. In Embodiment 49, the method of any one of embodiments 1 to 48, wherein the PKC inhibitor and the c-MET inhibitor are administered orally.
50. In Embodiment 50, the method of any one of embodiments 1 to 49, wherein method further comprises preserving the eye globe which had the ocular tumor.
51. In Embodiment 51, the method of any one of embodiments 1 to 50, wherein the method further comprises preserving a function of an eye which had the ocular tumor.
52. In Embodiment 52, the method of embodiment 51, wherein preserving the function of the eye which had the ocular tumor comprises at least partially preserving the eye function.
53. In Embodiment 53, the method of any one of embodiments 1 to 52, wherein method further comprises preventing irreversible vision loss.
54. In Embodiment 54, the method of any one of embodiments 1 to 52, wherein method further comprises preserving visual symptoms.
55. In Embodiment 55, the method of any one of embodiments 1 to 52, wherein method further comprises preserving depth perception.
56. In Embodiment 56, the method of any one of embodiments 1 to 55, wherein method further comprises preserving vision for greater than at least 1 year.
57. In Embodiment 57, the method of any one of embodiments 1 to 56, wherein method further comprises preventing metastasis.
58. In Embodiment 58, the method of any one of embodiments 1 to 56, wherein method further comprises delaying onset of metastasis.
59. In Embodiment 59, the method of any one of embodiments 1 to 58, wherein the ocular tumor is spatially separated from the optic nerve.
60. In Embodiment 60, the method of any one of embodiments 1 to 59, wherein the ocular tumor, prior to administering the neoadjuvant therapy, has a size from 5 mm to 19 mm LBD and less than 2.5 mm apical height.
61. In Embodiment 61, the method of any one of embodiments 1 to 59, wherein the ocular tumor, prior to administering the neoadjuvant therapy, has a size less than or equal to 19 mm LBD and 2.5-10 mm apical height.
62. In Embodiment 62, the method of any one of embodiments 1 to 59, wherein the ocular tumor, prior to administering the neoadjuvant therapy, has a size greater than 19 mm LBD.
63. In Embodiment 63, the method of any one of embodiments 1 to 59, wherein the ocular tumor, prior to administering the neoadjuvant therapy, has a size greater than 10 mm apical height and any size LBD.
64. In Embodiment 64, the method of any one of embodiments 1 to 58, wherein the ocular tumor, prior to administering the neoadjuvant therapy, has a size greater than 8 mm apical height and the tumor is located close to the optic nerve.
65. In Embodiment 65, the method of any one of embodiments 1 to 59, wherein the ocular tumor, prior to administering the neoadjuvant therapy, has a size of greater than 6 mm and less than 12 mm apical height, and not greater than 16 mm LBD.
66. In Embodiment 66, the method of any one of embodiments 1 to 59, wherein the ocular tumor, prior to administering the neoadjuvant therapy, has a size of greater than 3 mm and less than 8 mm apical height, and not greater than 16 mm LBD.
67. In Embodiment 67, the method of any one of embodiments 1 to 59, wherein the ocular tumor, prior to administering the neoadjuvant therapy, has a size of greater than 8 mm apical height, and greater than 16 mm LBD.
68. In Embodiment 68, the method of any one of embodiments 1 to 67, wherein the ocular tumor, prior to administering the neoadjuvant therapy, is not greater than 16 mm LBD.
69. In Embodiment 69, the method of any one of embodiments 1 to 68, wherein the size of the ocular tumor, prior to administering the neoadjuvant therapy, is greater than 10 mm in apical height.
70. In Embodiment 70, the method of any one of embodiments 1 to 69, wherein the size of the ocular tumor after administering the neoadjuvant therapy but prior to the interventional procedure is reduced by at least 20%.
71. In Embodiment 71, the method of any one of embodiments 1 to 70, wherein the size of the ocular tumor after administering the neoadjuvant therapy but prior to the interventional procedure is reduced by at least 30%.
72. In Embodiment 72, the method of any one of embodiments 1 to 71, wherein the size of the ocular tumor after administering the neoadjuvant therapy but prior to the interventional procedure is reduced by at least 50%.
73. In Embodiment 73, the method of any one of embodiments 1 to 72, wherein the PKC inhibitor, or an equivalent dose of a pharmaceutically acceptable salt thereof, is administered at a dose of about 100 to about 400 mg two times per day (BID).
74. In Embodiment 74, the method of any one of embodiments 1 to 73, wherein the PKC inhibitor, or an equivalent dose of a pharmaceutically acceptable salt thereof, is administered at a dose of about 100 mg BID, about 150 mg BID, about 200 mg BID, about 250 mg BID, about 300 mg BID, about 350 mg BID, or about 400 mg two times per day (BID).
75. In Embodiment 75, the method of any one of embodiments 1 to 74, wherein the PKC inhibitor, or an equivalent dose of a pharmaceutically acceptable salt thereof, is administered at a dose of about 200 to about 400 mg two times per day (BID).
76. In Embodiment 76, the method of any one of embodiments 1 to 75, wherein the PKC inhibitor, or an equivalent dose of a pharmaceutically acceptable salt thereof, is administered at a dose of about 300 mg two times per day (BID).
77. In Embodiment 77, the method of any one of embodiments 1 to 75, wherein the PKC inhibitor, or an equivalent dose of a pharmaceutically acceptable salt thereof, is administered at a dose of 200 mg two times per day (BID).
78. In Embodiment 78, the method of any one of embodiments 1 to 72, wherein the PKC inhibitor, or an equivalent dose of a pharmaceutically acceptable salt thereof, is administered at a dose of about 200 mg to about 600 mg daily.
79. In Embodiment 79, the method of any one of embodiments 1 to 78, wherein the c-MET inhibitor, or an equivalent dose of a pharmaceutically acceptable salt thereof, is administered at a dose of about 200 mg BID.
80. In Embodiment 80, the method of any one of embodiments 1 to 78, wherein the c-MET inhibitor, or an equivalent dose of a pharmaceutically acceptable salt thereof, is administered at a dose of about 250 mg BID.
81. In Embodiment 81, the method of any one of embodiments 1 to 72, wherein the PKC inhibitor, or an equivalent dose of a pharmaceutically acceptable salt thereof, is administered at a dose of about 400 mg to about 600 mg daily; and the c-MET inhibitor, or an equivalent dose of a pharmaceutically acceptable salt thereof, is administered at a dose of about 400 mg to about 500 mg daily.
82. In Embodiment 82, the method of any one of embodiments 1 to 81, wherein the PKC inhibitor, or an equivalent dose of a pharmaceutically acceptable salt thereof and the c-MET inhibitor, or an equivalent dose of a pharmaceutically acceptable salt thereof, are both administered daily for at least 7 days, at least 14 days, at least 21, days, at least 28 days, at least 56 days, at least 84 days, at least, 112 days, at last 140 days, or at least 168 days.
83. In Embodiment 83, the method of any one of embodiments 1 to 85, wherein the PKC inhibitor, or an equivalent dose of a pharmaceutically acceptable salt thereof, and c-MET inhibitor, or an equivalent dose of a pharmaceutically acceptable salt thereof, is administered daily for at least 7 days, at least 14 days, at least 21, days, or at least 28 days.
84. In Embodiment 84, the method of any one of embodiments 1 to 82, wherein the PKC inhibitor, or an equivalent dose of a pharmaceutically acceptable salt thereof, and c-MET inhibitor, or an equivalent dose of a pharmaceutically acceptable salt thereof, are both administered daily for at least 28 days, at least 56 days, at least 84 days, at least 140 days, or at least 168 days.
85. In Embodiment 85, the method of any one of embodiments 1 to 81, wherein the PKC inhibitor, or an equivalent dose of a pharmaceutically acceptable salt thereof, and c-MET inhibitor, or an equivalent dose of a pharmaceutically acceptable salt thereof, are both administered daily for at least 1 month, at least 2 months, at least 3 months, at least 4 months, at least 5 months or at least 6 months.
86. In Embodiment 86, the method of any one of embodiments 1 to 81, wherein the PKC inhibitor, or an equivalent dose of a pharmaceutically acceptable salt thereof, and c-MET inhibitor, or an equivalent dose of a pharmaceutically acceptable salt thereof, are both administered daily for about at least 1 month to at least about 6 months.
87. In Embodiment 87, the method of any one of embodiments 1 to 81, wherein the neoadjuvant therapy comprises
   (i) a dosing schedule comprising a first treatment cycle of at least one 7-day dosing cycle, wherein the PKC inhibitor, or an equivalent dose of a pharmaceutically acceptable salt thereof, is administered alone at a dose of about 300 mg BID every day of the first treatment cycle followed by
   (ii) a second treatment cycle of at least one 7-day dosing cycle, wherein the PKC inhibitor, or an equivalent dose of a pharmaceutically acceptable salt thereof, is administered at a dose of about 300 mg BID and the c-MET inhibitor, or an equivalent dose of a pharmaceutically acceptable salt thereof, is administered at a dose of about 200 mg BID every day of the second treatment cycle.
88. In Embodiment 88, the method of embodiment 87, wherein the second treatment cycle of at least one 7-day dosing cycle is followed by at least two or six 7-day dosing cycle wherein the PKC inhibitor, or an equivalent dose of a pharmaceutically acceptable salt thereof, is administered at a dose of about 300 mg BID and the c-MET inhibitor, or an equivalent dose of a pharmaceutically acceptable salt thereof, is administered at a dose of about 200 mg BID every day of the at least two or six 7-day dosing cycle.
89. In Embodiment 89, the method of any one of embodiments 1 to 81, wherein the adjuvant therapy comprises
   (i) a dosing schedule comprising a first treatment cycle of at least one 7-day dosing cycle, wherein the PKC inhibitor, or an equivalent dose of a pharmaceutically acceptable salt thereof, is administered alone at a dose of about 300 mg BID every day of the first treatment cycle followed by
   (ii) a second treatment cycle of at least one 7-day dosing cycle, wherein the PKC inhibitor, or an equivalent dose of a pharmaceutically acceptable salt thereof, is administered at a dose of about 300 mg BID and the c-MET inhibitor, or an equivalent dose of a pharmaceutically acceptable salt thereof, is administered at a dose of about 200 mg BID every day of the second treatment cycle.
90. In Embodiment 90, the method of embodiment 89, wherein the second treatment cycle of at least one 7-day dosing cycle is followed by at least two or six 7-day dosing cycle wherein the PKC inhibitor, or an equivalent dose of a pharmaceutically acceptable salt thereof, is administered at a dose of about 300 mg BID and the c-MET inhibitor, or an equivalent dose of a pharmaceutically acceptable salt thereof, is administered at a dose of about 200 mg BID every day of the at least two or six 7-day dosing cycle.
91. In Embodiment 91, the method of any one of embodiments 1 to 90, wherein the interventional procedure is selected from the group consisting of plaque brachytherapy (PBT), external beam radiotherapy (EBRT), stereotactic radiosurgery (SRS), and any combination thereof.
92. In Embodiment 92, the method of any one of embodiments 1 to 91, wherein the interventional procedure is plaque brachytherapy (PBT).
93. In Embodiment 93, the method of any one of embodiments 1 to 92, wherein the uveal melanoma is a solid tumor harboring GNAQ or GNA11 mutations.
94. In Embodiment 94, the method of any one of embodiments 1 to 93, wherein the patient has additional non-ocular tumor.
95. In Embodiment 95, the method of embodiment 94, wherein the non-ocular tumor is metastatic uveal melanoma.
96. In Embodiment 96, the method of any one of embodiments 1 to 95, wherein the ocular tumor is not malignant.
97. In Embodiment 97, the method of any one of embodiments 1 to 95, wherein the ocular tumor is malignant.
98. In Embodiment 98, the method of any one of embodiments 1 to 97, wherein the ocular tumor is intraocular tumor.
99. In Embodiment 99, the method of any one of embodiments 1 to 98, wherein the patient is human leukocyte antigen (HLA)-A2 negative.
100. In Embodiment 100, the method of any one of embodiments 1 to 98, wherein the patient is human leukocyte antigen (HLA)-A2 positive.
101. In Embodiment 101, the method of any one of embodiments 1 to 100, wherein the patient has not been previously treated with a PKC inhibitor and/or a c-MET inhibitor.
102. In Embodiment 102, the method of any one of above embodiments, wherein the size of the ocular tumor is measured using RECIST 1.1 criteria.
103. In Embodiment 103, the method of any one of above embodiments, wherein the size of the ocular tumor is determined by CT scan, MRI, or ultrasound, or a combination thereof.
104. In Embodiment 104, the method of any one of above embodiments, wherein the size of the ocular tumor is determined by CT scan.
105. In Embodiment 105, the method of any one of above embodiments, wherein the size of the ocular tumor is determined by MRI.
106. In Embodiment 106, the method of any one of above embodiments, wherein the size of the ocular tumor is determined by ultrasound.
107. In Embodiment 107, the method of any one of above embodiments, wherein the neoadjuvant therapy is administered to the patient for at least 1 month, at least 2 months, at least 3 months, at least 4 months, at least 5 months, at least 6 months or at least 7 months.
108. In Embodiment 108, the method of any one of above embodiments, wherein the adjuvant therapy is administered to the patient for at least 1 month, at least 2 months, at least 3 months, at least 4 months, at least 5 months, at least 6 months or at least 7 months.

### Non-limiting Exemplary Embodiments

1. In Embodiment 1 is a method of treating uveal melanoma in a patient having an ocular tumor, the method comprising
   a) treating the ocular tumor with an interventional procedure, and
   b) administering a PKC inhibitor and administering a c-MET inhibitor to the patient as an adjuvant therapy.
2. In Embodiment 2 is the method of embodiment 1, wherein the ocular tumor is defined by a set of dimensions comprising (i) greater than or equal to 5 mm largest basal diameter (LBD) and any apical height, or (ii) any LBD and greater than 3 mm apical height.
3. In embodiment 3 is a method of treating a patient having uveal melanoma with an ocular tumor with a size indicated for an interventional procedure comprising enucleation of the eye, the method comprising
   a) treating the ocular tumor with an interventional procedure other than enucleation, and
   b) administering a PKC inhibitor and administering a c-MET inhibitor to the patient as an adjuvant therapy.
4. In Embodiment 4 is a method of treating a patient having uveal melanoma with an ocular tumor with a size indicated for an interventional procedure comprising irradiating the ocular tumor, the method comprising
   a) treating the ocular tumor with an interventional procedure other than enucleation, and
   b) administering a PKC inhibitor and administering a c-MET inhibitor to the patient as an adjuvant therapy.
5. In Embodiment 5 is a method of treating uveal melanoma in a patient having an ocular tumor with a size defined by a set of dimensions comprising (i) greater than 16 mm LBD and any apical height; (ii) any LBD and greater than 8 mm apical height; or (iii) less than 16 mm LBD and 3-8 mm apical height, the method comprising
   a) treating the ocular tumor with an interventional procedure, and
   b) administering a PKC inhibitor and administering a c-MET inhibitor to the patient as an adjuvant therapy.
6. In Embodiment 6 is a method of treating uveal melanoma in a patient having an ocular tumor with a size defined by a set of dimensions comprising (i) greater than 16 mm LBD and any apical height, or (ii) any LBD and greater than 8 mm apical height, the method comprising
   a) treating the ocular tumor with an interventional procedure, and
   b) administering a PKC inhibitor and administering a c-MET inhibitor to the patient as an adjuvant therapy.
7. In Embodiment 7 is a method of treating uveal melanoma in a patient having an ocular tumor with a size defined by a set of dimensions comprising (i) greater than 19 mm LBD and any apical height, (ii) any LBD and greater than 10 mm apical height, (iii) greater than 8 mm apical height, any LBD, and the tumor is located close to the optic nerve, or (iv) less than or equal to 19 mm LBD and 2.5-10 mm apical height, the method comprising
   a) treating the ocular tumor with an interventional procedure, and
   b) administering a PKC inhibitor and administering a c-MET inhibitor to the patient as an adjuvant therapy.
8 In Embodiment 8 is a method of treating uveal melanoma in a patient having an ocular tumor with a size defined by a set of dimensions comprising (i) greater than 19 mm LBD and any apical height, (ii) any LBD and greater than 10 mm apical height, (iii) greater than 8 mm apical height, any LBD, and the tumor is located close to the optic nerve, or (iv) less than or equal to 19 mm LBD and 2.5-10 mm apical height, the method comprising
   a) treating the ocular tumor with an interventional procedure, and
   b) administering a PKC inhibitor and administering a c-MET inhibitor to the patient as an adjuvant therapy.
9. In Embodiment 9 is a method of treating uveal melanoma in a patient having an ocular tumor with a size defined by a set of dimensions comprising (i) up to 22 mm in LBD and (ii) up to 15 mm in apical height, the method comprising
   a) treating the ocular tumor with an interventional procedure, and
   b) administering a PKC inhibitor and administering a c-MET inhibitor to the patient as an adjuvant therapy.
10. In Embodiment 10 is a method of treating uveal melanoma in a patient having an ocular tumor with a size defined by a set of dimensions comprising (i) up to 22 mm in LBD or (ii) up to 15 mm in apical height, the method comprising
   a) treating the ocular tumor with an interventional procedure, and
   b) administering a PKC inhibitor and administering a c-MET inhibitor to the patient as an adjuvant therapy.
11. In Embodiment 11 is a method of treating uveal melanoma in a patient having an ocular tumor with a size defined by a set of dimensions comprising (i) at least 6 mm in LBD and (ii) at least 3 mm in apical height, the method comprising
   a) treating the ocular tumor with an interventional procedure, and
   b) administering a PKC inhibitor and administering a c-MET inhibitor to the patient as an adjuvant therapy.
12. In Embodiment 12 is a method of treating uveal melanoma in a patient having an ocular tumor with a size defined by a set of dimensions comprising (i) at least 6 mm in LBD or (ii) at least 3 mm in apical height, the method comprising
   a) treating the ocular tumor with an interventional procedure, and
   b) administering a PKC inhibitor and administering a c-MET inhibitor to the patient as an adjuvant therapy.
13. In Embodiment 13, the method of Embodiment 12, wherein the patients having sub-foveal or greater than 180-degree optic nerve involved tumors were excluded.
14. In Embodiment 14 is a method of treating uveal melanoma in a patient having an ocular tumor with a size defined by a set of dimensions comprising (i) greater than 19 mm LBD and any apical height, (ii) any LBD and greater than 10 mm apical height, or (iii) greater than 8 mm apical height, any LBD, and the tumor is located close to the optic nerve, the method comprising
   a) treating the ocular tumor with an interventional procedure, and
   b) administering a PKC inhibitor and administering a c-MET inhibitor to the patient as an adjuvant therapy.
15. In Embodiment 15, the method of any one of embodiments 1 to 14, wherein the interventional procedure comprises local surgical resection of the tumor, irradiating the tumor, or any combination thereof.
16. In Embodiment 16, the method of any one of embodiments 1 to 14, the interventional procedure comprises local surgical resection of the tumor.
17. In Embodiment 17, the method of any one of embodiments 1 to 14, wherein the interventional procedure comprises irradiating the tumor.
18. In Embodiment 18, the method of any one of embodiments 1 to 14, wherein the interventional procedure is irradiating the tumor.
19. In Embodiment 19, the method of any one of embodiments 1 to 18, wherein the PKC inhibitor is Compound 1:

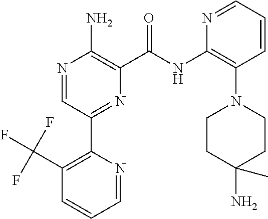 [SMILES: CC1(N)CCN(c2cccnc2NC(=O)c2nc(-c3ncccc3C(F)(F)F)cnc2N)CC1]

or a pharmaceutically acceptable salt thereof.
20. In Embodiment 20, the method of any one of embodiments 1 to 19, wherein the PKC inhibitor is Compound 1.
21. In Embodiment 21, the method of any one of embodiments 1 to 20, wherein the c-MET inhibitor is Compound 2:

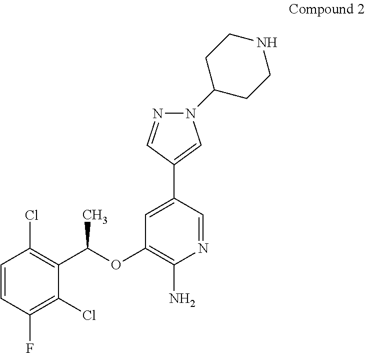 [SMILES: C[C@@H](Oc1cc(-c2cnn(C3CCNCC3)c2)cnc1N)c1c(Cl)ccc(F)c1Cl]

or a pharmaceutically acceptable salt thereof.
22. In Embodiment 22, the method of any one of embodiments 1 to 21, wherein the c-MET inhibitor is Compound 2.
23. In Embodiment 23, the method of any one of embodiments 1 to 22, wherein the interventional procedure is a globe preserving treatment (GPT).
24. In Embodiment 24, the method of any one of embodiments 1 to 23, wherein the interventional procedure is selected from the group consisting of plaque brachytherapy (PBT), external beam radiotherapy (EBRT), stereotactic radiosurgery (SRS), LASER, additional pharmacological agent therapy, and any combination thereof.
25. In Embodiment 25, the method of any one of embodiments 1 to 23, wherein the globe preserving treatment is selected from the group consisting of limited surgical resection or ablation (LSRA), radiation therapy (RT), brachytherapy (BT), external beam radiotherapy (EBRT), stereotactic radiosurgery (SRS), radiation therapy alone (RTA), radiation therapy and supplemental laser therapy (RT+SLT), and any combination thereof.
26. In Embodiment 26, the method of any one of embodiments 1 to 23, wherein the interventional procedure is selected from the group consisting of proton beam radiotherapy, transpupillary thermotherapy (TTT), thermotherapy, laser photocoagulation therapy, limited surgical resection or ablation (LSRA), radiation therapy (RT), brachytherapy (BT), external beam radiotherapy (EBRT), stereotactic radiosurgery (SRS), radiation therapy alone (RTA), radiation therapy and supplemental laser therapy (RT+SLT), charged-particles beam radiotherapy, and any combination thereof.
27. In Embodiment 27, the method of any one of embodiments 1 to 23, wherein the interventional procedure is brachytherapy (BT).
28. In Embodiment 28, the method of any one of embodiments 24 to 26, wherein stereotactic radiotherapy (SRS) is selected from the group consisting of gamma-knife, cyber knife, and linear accelerator (LINAC),
29. In Embodiment 29, the method of any one of embodiments 1 to 28, wherein the method comprises administering a PKC inhibitor and administering a c-MET inhibitor to the patient as an adjuvant therapy during the period of the interventional procedure.
30. In Embodiment 30, the method of any one of embodiments 1 to 28, wherein the method comprises administering a PKC inhibitor and administering a c-MET inhibitor to the patient as an adjuvant therapy after the completion of the interventional procedure.
31. In Embodiment 31, the method of Embodiment 30, wherein the adjuvant therapy is administered at least 1 week, at least 2 weeks, at least 3 weeks, at least 4 weeks, at least 5 weeks, or at least 6 weeks after the completion of the interventional procedure.
32. In Embodiment 32, the method of Embodiment 30 or 31, wherein the adjuvant therapy is administered at least 4 weeks, at least 5 weeks, or at least 6 weeks after the completion of the interventional procedure.
33. In Embodiment 33, the method of any one of embodiments 1 to 32, wherein the PKC inhibitor and the c-MET inhibitor are administered orally.
34. In Embodiment 34, the method of any one of embodiments 1 to 33, wherein method further comprises preserving the eye globe which had the ocular tumor.
35. In Embodiment 35, the method of any one of embodiments 1 to 34, wherein the method further comprises preserving a function of an eye which had the ocular tumor.
36. In Embodiment 36, the method of embodiment 35, wherein preserving the function of the eye which had the ocular tumor comprises at least partially preserving the eye function.
37. In Embodiment 37, the method of any one of embodiments 1 to 36, wherein method further comprises preventing irreversible vision loss.
38. In Embodiment 38, the method of any one of embodiments 1 to 37, wherein method further comprises preserving visual symptoms.
39. In Embodiment 39, the method of any one of embodiments 1 to 38, wherein method further comprises preserving depth perception.
40. In Embodiment 40, the method of any one of embodiments 1 to 39, wherein method further comprises preserving vision for greater than at least 1 year.
41. In Embodiment 41, the method of any one of embodiments 1 to 40, wherein method further comprises preventing metastasis.
42. In Embodiment 42, the method of any one of embodiments 1 to 40, wherein method further comprises delaying onset of metastasis.
43. In Embodiment 43, the method of any one of embodiments 1 to 42, wherein the ocular tumor is spatially separated from the optic nerve.
44. In Embodiment 44, the method of any one of embodiments 1 to 43, wherein the ocular tumor, prior to the interventional procedure, has a size from 5 mm to 19 mm LBD and less than 2.5 mm apical height.
45. In Embodiment 45, the method of any one of embodiments 1 to 43, wherein the ocular tumor, prior to the interventional procedure, has a size less than or equal to 19 mm LBD and 2.5-10 mm apical height.
46. In Embodiment 46, the method of any one of embodiments 1 to 43, wherein the ocular tumor, prior to the interventional procedure, has a size greater than 19 mm LBD.
47. In Embodiment 47, the method of any one of embodiments 1 to 43, wherein the ocular tumor, prior to the interventional procedure, has a size greater than 10 mm apical height and any size LBD.
48. In Embodiment 48, the method of any one of embodiments 1 to 43, wherein the ocular tumor, prior to the interventional procedure, has a size greater than 8 mm apical height and the tumor is located close to the optic nerve.
49. In Embodiment 49, the method of any one of embodiments 1 to 43, wherein the ocular tumor, prior to the interventional procedure, has a size of greater than 6 mm and less than 12 mm apical height, and not greater than 16 mm LBD.
50. In Embodiment 50, the method of any one of embodiments 1 to 43, wherein the ocular tumor, prior to the interventional procedure, has a size of greater than 3 mm and less than 8 mm apical height, and not greater than 16 mm LBD.
51. In Embodiment 51, the method of any one of embodiments 1 to 43, wherein the ocular tumor, prior to the interventional procedure, has a size of greater than 8 mm apical height, and greater than 16 mm LBD.
52. In Embodiment 52, the method of any one of embodiments 1 to 43, wherein the ocular tumor, prior to the interventional procedure, is not greater than 16 mm LBD.
53. In Embodiment 53, the method of any one of embodiments 1 to 43, wherein the size of the ocular tumor, prior to the interventional procedure, is greater than 10 mm in apical height.
54. In Embodiment 54, the method of any one of embodiments 1 to 53, wherein the size of the ocular tumor after the interventional procedure is reduced by at least 10%.
55. In Embodiment 55, the method of any one of embodiments 1 to 54, wherein the size of the ocular tumor after the interventional procedure is reduced by at least 20%.
56. In Embodiment 56, the method of any one of embodiments 1 to 55, wherein the size of 52 the ocular tumor after the interventional procedure is reduced by at least 30%.
57. In Embodiment 57, the method of any one of embodiments 1 to 56, wherein the size of the ocular tumor after the interventional procedure is reduced by at least 50%.
58. In Embodiment 58, the method of any one of embodiments 1 to 57, wherein the PKC inhibitor, or an equivalent dose of a pharmaceutically acceptable salt thereof, is administered at a dose of about 100 to about 400 mg two times per day (BID).
59. In Embodiment 59, the method of any one of embodiments 1 to 58, wherein the PKC inhibitor, or an equivalent dose of a pharmaceutically acceptable salt thereof, is administered at a dose of about 100 mg BID, about 150 mg BID, about 200 mg BID, about 250 mg BID, about 300 mg BID, about 350 mg BID, or about 400 mg two times per day (BID).
60. In Embodiment 60, the method of any one of embodiments 1 to 59, wherein the PKC inhibitor, or an equivalent dose of a pharmaceutically acceptable salt thereof, is administered at a dose of about 200 to about 400 mg two times per day (BID).
61. In Embodiment 61, the method of any one of embodiments 1 to 60, wherein the PKC inhibitor, or an equivalent dose of a pharmaceutically acceptable salt thereof, is administered at a dose of about 300 mg two times per day (BID).
62. In Embodiment 62, the method of any one of embodiments 1 to 57, wherein the PKC inhibitor, or an equivalent dose of a pharmaceutically acceptable salt thereof, is administered at a dose of about 400 mg to about 600 mg daily.
63. In Embodiment 63, the method of any one of embodiments 1 to 57, wherein the PKC inhibitor, or an equivalent dose of a pharmaceutically acceptable salt thereof, is administered at a dose of about 200 mg to about 600 mg daily.
64. In Embodiment 64, the method of any one of embodiments 1 to 63, wherein the c-MET inhibitor, or an equivalent dose of a pharmaceutically acceptable salt thereof, is administered at a dose of about 200 mg BID.
65. In Embodiment 65, the method of any one of embodiments 1 to 63, wherein the c-MET inhibitor, or an equivalent dose of a pharmaceutically acceptable salt thereof, is administered at a dose of about 250 mg BID.
66. In Embodiment 66, the method of any one of embodiments 1 to 57, wherein the PKC inhibitor, or an equivalent dose of a pharmaceutically acceptable salt thereof, is administered at a dose of about 400 mg to about 600 mg daily; and the c-MET inhibitor, or an equivalent dose of a pharmaceutically acceptable salt thereof, is administered at a dose of about 400 mg to about 500 mg daily.
67. In Embodiment 67, the method of any one of embodiments 1 to 66, wherein the PKC inhibitor, or an equivalent dose of a pharmaceutically acceptable salt thereof, and the c-MET inhibitor, or an equivalent dose of a pharmaceutically acceptable salt thereof, are both administered daily for at least 7 days, at least 14 days, at least 21, days, at least 28 days, at least 56 days, at least 84 days, at least, 112 days, at last 140 days, or at least 168 days.
68. In Embodiment 68, the method of any one of embodiments 1 to 67, wherein the PKC inhibitor, or an equivalent dose of a pharmaceutically acceptable salt thereof, and the c-MET inhibitor, or an equivalent dose of a pharmaceutically acceptable salt thereof, are both administered daily for at least 7 days, at least 14 days, at least 21, days, or at least 28 days.
69. In Embodiment 69, the method of any one of embodiments 1 to 67, wherein the PKC inhibitor, or an equivalent dose of a pharmaceutically acceptable salt thereof, and the c-MET inhibitor, or an equivalent dose of a pharmaceutically acceptable salt thereof, are both administered daily for at least 28 days, at least 56 days, at least 84 days, at least 140 days, or at least 168 days.
70. In Embodiment 70, the method of any one of embodiments 1 to 66, wherein the PKC inhibitor, or an equivalent dose of a pharmaceutically acceptable salt thereof, and the c-MET inhibitor, or an equivalent dose of a pharmaceutically acceptable salt thereof, are both administered daily for at least 1 month, at least 2 months, at least 3 months, at least 4 months, at least 5 months or at least 6 months.
71. In Embodiment 71, the method of any one of embodiments 1 to 66, wherein the adjuvant therapy comprises
   (i) a dosing schedule comprising a first treatment cycle of at least one 7-day dosing cycle, wherein the PKC inhibitor, or an equivalent dose of a pharmaceutically acceptable salt thereof, is administered alone at a dose of about 300 mg BID every day of the first treatment cycle followed by
   (ii) a second treatment cycle of at least one 7-day dosing cycle, wherein the PKC inhibitor, or an equivalent dose of a pharmaceutically acceptable salt thereof, is administered at a dose of about 300 mg BID and the c-MET inhibitor, or an equivalent dose of a pharmaceutically acceptable salt thereof, is administered at a dose of about 200 mg BID every day of the second treatment cycle.
72. In Embodiment 72, the method of embodiment 71, wherein the second treatment cycle of at least one 7-day dosing cycle is followed by at least two or six 7-day dosing cycle wherein the PKC inhibitor, or an equivalent dose of a pharmaceutically acceptable salt thereof, is administered at a dose of about 300 mg BID and the c-MET inhibitor, or an equivalent dose of a pharmaceutically acceptable salt thereof, is administered at a dose of about 200 mg BID every day of the at least two or six 7-day dosing cycle.
73. In Embodiment 73, the method of any one of embodiments 1 to 72, wherein the interventional procedure is selected from the group consisting of plaque brachytherapy (PBT), external beam radiotherapy (EBRT), stereotactic radiosurgery (SRS), and any combination thereof.
74. In Embodiment 74, the method of any one of embodiments 1 to 73, wherein the interventional procedure is plaque brachytherapy (PBT).
75. In Embodiment 75, the method of any one of embodiments 1 to 74, wherein the uveal melanoma is a solid tumor harboring GNAQ or GNA11 mutations.
76. In Embodiment 76, the method of any one of embodiments 1 to 75, wherein the patient has additional non-ocular tumor.
77. In Embodiment 77, the method of embodiment 76, wherein the non-ocular tumor is metastatic uveal melanoma.
78. In Embodiment 78, the method of any one of embodiments 1 to 77, wherein the ocular tumor is not malignant.
79. In Embodiment 79, the method of any one of embodiments 1 to 77, wherein the ocular tumor is malignant.
80. In Embodiment 80, the method of any one of embodiments 1 to 79, wherein the ocular tumor is intraocular tumor.
81. In Embodiment 81, the method of any one of embodiments 1 to 80, wherein the patient is human leukocyte antigen (HLA)-A2 negative.
82. In Embodiment 82, the method of any one of embodiments 1 to 80, wherein the patient is human leukocyte antigen (HLA)-A2 positive.
83. In Embodiment 83, the method of any one of embodiments 1 to 82, wherein the patient has not been previously treated with a PKC inhibitor and/or a c-MET inhibitor.
84. In Embodiment 84, the method of any one of above embodiments, wherein the size of the ocular tumor is measured using RECIST 1.1 criteria.
85. In Embodiment 85, the method of any one of above embodiments, wherein the size of the ocular tumor is determined by CT scan, MRI, or ultrasound, or a combination thereof.
86. In Embodiment 86, the method of any one of above embodiments, wherein the size of the ocular tumor is determined by CT scan.
87. In Embodiment 87, the method of any one of above embodiments, wherein the size of the ocular tumor is determined by MRI.
88. In Embodiment 88, the method of any of above embodiments, wherein the size of the ocular tumor is determined by ultrasound.

### Non-limiting Embodiments

1. A method of treating uveal melanoma in a patient having an ocular tumor, the method comprising
   a) administering a PKC inhibitor and administering a c-MET inhibitor to the patient as a neoadjuvant therapy;
   b) reducing the size of the ocular tumor by at least 10%; and
   c) treating the reduced-size ocular tumor with an interventional procedure.
2. The method of embodiment 1, wherein the ocular tumor is defined by a set of dimensions comprising (i) greater than or equal to 5 mm largest basal diameter (LBD) and any apical height, or (ii) any LBD and greater than 3 mm apical height.
3. A method of treating a patient having uveal melanoma with an ocular tumor with a size indicated for an interventional procedure comprising enucleation of the eye, the method comprising
   a) administering a PKC inhibitor and administering a c-MET inhibitor to the patient as a neoadjuvant therapy;
   b) reducing the size of the ocular tumor by at least 10%; and
   c) treating the reduced-size ocular tumor with an interventional procedure other than enucleation.
4. The method of embodiment 3, wherein the ocular tumor is defined by a set of dimensions comprising (i) up to 22 mm in LBD and/or (ii) up to 15 mm in apical height.
5. A method of treating a patient having uveal melanoma with an ocular tumor with a size indicated for an interventional procedure comprising irradiating the ocular tumor, the method comprising
   a) administering a PKC inhibitor and administering a c-MET inhibitor to the patient as a neoadjuvant therapy;
   b) reducing the size of the ocular tumor by at least 10%; and
   c) treating the reduced-size ocular tumor with an interventional procedure other than enucleation.
6. The method of embodiment 5, wherein the ocular tumor is defined by a set of dimensions comprising (i) at least 6 mm in LBD and/or (ii) at least 3 mm in apical height.
7. The method of any one of embodiments 1 to 6, wherein the interventional procedure comprises local surgical resection of the tumor, irradiating the tumor, or any combination thereof.
8. The method of any one of embodiments 1 to 7, wherein the interventional procedure comprises irradiating the tumor.
9. A method of treating uveal melanoma in a patient having an ocular tumor, the method comprising:
   a) determining a first therapeutically effective dose of radiation for treating the ocular tumor;
   b) administering a PKC inhibitor and administering a c-MET inhibitor to the patient as a neoadjuvant therapy;
   c) reducing the size of the ocular tumor; and
   d) irradiating the reduced-size ocular tumor with a second therapeutically effective dose of radiation less than the first therapeutically effective dose of radiation.
10. The method of embodiment 9, wherein in step (c) the ocular tumor is reduced by at least 10%.
11. The method of embodiment 9 or 10, wherein the ocular tumor before step (a) is defined by a set of dimensions comprising (i) at least 6 mm in LBD and/or (ii) at least 3 mm in apical height.
12. The method of any one of embodiments 9 to 11, wherein in step (d) the second therapeutically effective dose of radiation is at least 10% less than the first therapeutically effective dose of radiation.
13. The method of any one of embodiments 1 to 12, wherein the PKC inhibitor is Compound 1:

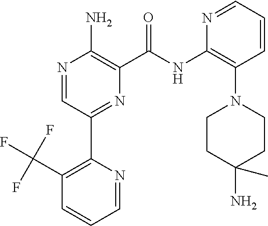 [SMILES: CC1(N)CCN(c2cccnc2NC(=O)c2nc(-c3ncccc3C(F)(F)F)cnc2N)CC1]

or a pharmaceutically acceptable salt thereof.
14. The method of any one of embodiments 1 to 13, wherein the c-MET inhibitor is Compound 2:

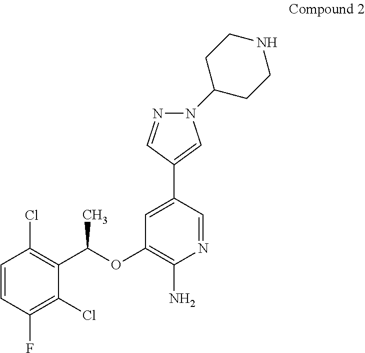 [SMILES: C[C@@H](Oc1cc(-c2cnn(C3CCNCC3)c2)cnc1N)c1c(Cl)ccc(F)c1Cl]

or a pharmaceutically acceptable salt thereof.
15. The method of any one of embodiments 1 to 14, wherein the interventional procedure is a globe preserving treatment (GPT).
16. The method of any one of embodiments 1 to 15, wherein the interventional procedure is selected from the group consisting of plaque brachytherapy (PBT), external beam radiotherapy (EBRT), stereotactic radiosurgery (SRS), and any combination thereof.
17. The method of any one of embodiments 1 to 16, wherein the interventional procedure is plaque brachytherapy (PBT).
18. The method of any one of claim embodiments 1 to 15, wherein the globe preserving treatment is selected from the group consisting of limited surgical resection or ablation (LSRA), radiation therapy (RT), brachytherapy (BT), external beam radiotherapy (EBRT), stereotactic radiosurgery (SRS), radiation therapy alone (RTA), radiation therapy and supplemental laser therapy (RT+SLT), and any combination thereof.
19. The method of any one of embodiments 1 to 17, wherein the method further comprises continuing administering a PKC inhibitor and administering a c-MET inhibitor to the patient as an adjuvant therapy during the period of the interventional procedure.
20. The method of any one of embodiments 1 to 17, wherein the method further comprises continuing administering a PKC inhibitor and administering a c-MET inhibitor to the patient as an adjuvant therapy after the completion of the interventional procedure.
21. The method of embodiment 20, wherein the adjuvant therapy is administered at least 4 weeks, at least 5 weeks, or at least 6 weeks after the completion of the interventional procedure.
22. The method of any one of claims 1 to 21, wherein the PKC inhibitor and the c-MET inhibitor are both administered orally.
23. The method of any one of embodiments 1 to 22, wherein the method further comprises preserving the eye globe which had the ocular tumor.
24. The method of any one of embodiments 1 to 23, wherein the method further comprises preserving a function of the eye which had the ocular tumor.
25. The method of embodiment 24, wherein preserving the function of the eye comprises at least partially preserving the eye function.
26. The method of any one of embodiments 1 to 25, wherein the method further comprises preventing irreversible vision loss.
27. The method of any one of embodiments 1 to 25, wherein the method further comprises preserving visual symptoms.
28. The method of any one of embodiments 1 to 25, wherein the method further comprises preserving depth perception.
29. The method of any one of embodiments 1 to 28, wherein the method further comprises preserving vision for greater than at least 1 year.
30. The method of any one of embodiments 1 to 29, wherein the method further comprises preventing metastasis.
31. The method of any one of embodiments 1 to 29, wherein the method further comprises delaying onset of metastasis.
32. The method of any one of embodiments 1 to 31, wherein the ocular tumor is spatially separated from the optic nerve.
33. The method of any one of embodiments 1 to 32, wherein the ocular tumor, prior to administering the neoadjuvant therapy, has a size
   (i) from 5 mm to 19 mm LBD and less than 2.5 mm apical height,
   ii) less than or equal to 19 mm LBD and 2.5-10 mm apical height,
   iii) greater than 19 mm LBD,
   iv) greater than 10 mm apical height and any size LBD,
   v) greater than 8 mm apical height and the tumor is located close to the optic nerve,
   vi) greater than 6 mm and less than 12 mm apical height, and not greater than 16 mm LBD,
   vii) greater than 3 mm and less than 8 mm apical height, and not greater than 16 mm LBD,
   viii) greater than 8 mm apical height, and greater than 16 mm LBD,
   ix) not greater than 16 mm LBD, or
   x) greater than 10 mm in apical height.
34. The method of any one of embodiments 1 to 33, wherein the size of the ocular tumor after administering the neoadjuvant therapy but prior to the interventional procedure is reduced by at least 20%.
35. The method of any one of embodiments 1 to 34, wherein the PKC inhibitor, or an equivalent dose of a pharmaceutically acceptable salt thereof, is administered at a dose of about 200 mg to about 400 mg two times per day (BID).
36. The method of any one of embodiments 1 to 35, wherein the PKC inhibitor, or an equivalent dose of a pharmaceutically acceptable salt thereof, is administered at a dose of about 300 mg two times per day (BID).
37. The method of any one of embodiments 1 to 36, wherein the c-MET inhibitor, or an equivalent dose of a pharmaceutically acceptable salt thereof, is administered at a dose of about 200 mg BID.
38. The method of any one of embodiments 1 to 34, wherein the PKC inhibitor, or an equivalent dose of a pharmaceutically acceptable salt thereof, is administered at a dose of about 400 mg to about 600 mg daily; and the c-MET inhibitor, or an equivalent dose of a pharmaceutically acceptable salt thereof, is administered at a dose of about 400 mg to about 500 mg daily.
39. The method of any one of embodiments 1 to 38, wherein the PKC inhibitor, or an equivalent dose of a pharmaceutically acceptable salt thereof and the c-MET inhibitor, or an equivalent dose of a pharmaceutically acceptable salt thereof, are both administered daily for at least 7 days, at least 14 days, at least 21, days, at least 28 days, at least 56 days, at least 84 days, at least, 112 days, at last 140 days, or at least 168 days.
40. The method of any one of embodiments 1 to 39, wherein the uveal melanoma is a solid tumor harboring GNAQ or GNA11 mutations.
41. The method of any one of embodiments 1 to 41, wherein the ocular tumor is intraocular tumor.
42. The method of any one of embodiments 1 to 41, wherein the size of the ocular tumor is measured using RECIST 1.1 criteria.
43. The method of any one of embodiments 1 to 42, wherein the size of the ocular tumor is determined by CT scan, MRI, or ultrasound, or a combination thereof.
44. The method of any one of embodiments 1 to 38 and 40 to 43, wherein the neoadjuvant therapy is administered to the patient for at least 1 month, at least 2 months, at least 3 months, at least 4 months, at least 5 months, or at least 6 months.
45. The method of any one of embodiments 1 to 38 and 40 to 44, wherein the adjuvant therapy is administered to the patient for at least 1 month, at least 2 months, at least 3 months, at least 4 months, at least 5 months, or at least 6 months.

Those skilled in the art will recognize, or be able to ascertain using no more than routine experimentation, numerous equivalents to the specific procedures, embodiments, claims, and examples described herein. Such equivalents were considered to be within the scope of this disclosure and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present disclosure. Moreover, all values that fall within these ranges, as well as the upper or lower limits of a range of values, are also contemplated by the present application.

The following examples further illustrate aspects of the present disclosure. However, they are in no way a limitation of the teachings of the present disclosure as set forth.

### EXAMPLES

The compounds and methods disclosed herein are further illustrated by the following examples, which should not be construed as further limiting. The practice of the present disclosure will employ, unless otherwise indicated, conventional techniques of organic synthesis, cell biology, cell culture, and molecular biology, which are within the skill of the art.

Processes for preparing the compounds disclosed herein can be found, at least, in WO 2016/020864 and WO 2006/021884, the contents of which are incorporated in their entirety.

### Example 1. Clinical Protocol

### Selected Eligibility Criteria:

### Inclusion:

For example, clinically diagnosed Uveal (not iris) Melanoma of >6 mm to <12 mm apical height, not greater than 16 mm LBD and not abutting the optic nerve. Additional exemplary criteria related to ocular tumor size in a patient before the treatment with Compound 1 and Compound 2 as described herein are listed throughout the application.
ECOG 0-1
Adequate Organ functioning at screening including liver, kidney, cardiac function

### Exclusion:

Patients can be excluded if they have contraindication to the use of Compound 1 or Compound 2 including allergies or clinical contraindications to either drug such as pre-existing bleeding diathesis or malabsorption syndromes
In addition, given the potential for additive toxicity, patients with a history of interstitial lung disease/pneumonitis or a recent history of syncope can be excluded
Recent surgery or radiotherapy
Prior gastrectomy or upper bowel removal or any other gastrointestinal disorder or defect that may affect absorption
Females who are pregnant or breastfeeding
Impaired cardiac function or recent history of ischemic cardiac events
Treatment with prohibited medications that cannot be discontinued prior to study entry

### Example 2. Compound 1 and Compound 2 Partial Responses in the Uvea

A first-line MUM patient (NCT03947385) also having uveal melanoma (intact primary ocular tumor) received Compound 1 (300 mg BID) and Compound 2 (200 mg BID) for about eleven months. Approximately 10 months after receiving treatment of (Compound 1 (300 mg BID) and Compound 2 (200 mg BID)), the patient's ocular tumor is scored as absent by RECIST 1.1, and with an observed 81% reduction of apical tumor height by ultrasound relative to baseline (prior to starting therapy). Visual symptoms were also resolved with treatment.

A MUM patient (Patient 2) with an intact primary lesion (intact ocular tumor which had received radiotherapy treatment prior to entering the study) received Compound 1 (300 mg BID) and Compound 2 (200 mg BID) for eleven months and 2 weeks. A pre-study baseline scan was performed 5 months before the patient received the combination. The patient received 7 months of treatment with the combination and a reduction in the apical height of 14.8% and 16.6% largest basal diameter of the ocular lesion was observed based on scan.

A UM patient (Patient 3) with an intact primary lesion (intact ocular tumor) received Compound 1 (300 mg BID) and Compound 2 (200 mg BID). The patient received 4 months of treatment with the combination and a reduction in the apical height of 79% of the ocular lesion was observed based on scan. The patient remained on treatment for additional 2 months with the combination and a reduction in the apical height of 84% and 22% reduction in the largest basal diameter of the ocular lesion was observed based on scan (B-scan ultrasonography). All ocular lesion reduction measurements were based on baseline ocular lesion, which is the ocular tumor measurements before any treatment with Compound 1 and/or Compound 2. The patient was referred for enucleation before entering the study and through treatment with the combination enucleation was avoided and the eye was preserved. The patient had a pre-treatment score of 6/120 (6/60=legally blind) and post-treatment vision score of 6/5 after replacement of an intraocular lens for a cataract. After replacement of intraocular lens, the patient was treated with Ruthenium plaque brachytherapy, which was planned with Plaque Simulator (version 6.9.2, Eye Physics, LLC, Los Alamitos, CA, USA) using a 20 mm round CCB plaque (Eckert & Ziegler BEBIG GmbH, Berlin, Germany). The plaque delivered 350 Gy to the outer sclera, resulting in a tumour apex dose of 141.41 Gy, a macula dose of 4.55 Gy and an optic disc dose of 14.51. One week after plaque placement, the patient's eye visual acuity was 6/9. The patient continues on adjuvant therapy of Compound 1 ((300 mg BID) and Compound 2 (200 mg BID).

All ocular lesion reduction measurements were based on baseline ocular lesion, which is the ocular tumor measurements before any treatment with Compound 1 and/or Compound 2.

Particular embodiments of this invention are described herein, including the best mode known to the inventors for carrying out the invention. Upon reading the foregoing, description, variations of the disclosed embodiments may become apparent to individuals working in the art, and it is expected that those skilled artisans may employ such variations as appropriate. Accordingly, it is intended that the invention be practiced otherwise than as specifically described herein, and that the invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

All patent applications, patents, and printed publications cited herein are incorporated herein by reference in the entireties, except for any definitions, subject matter disclaimers or disavowals, and except to the extent that the incorporated material is inconsistent with the express disclosure herein, in which case the language in this disclosure controls.

1. A method of treating uveal melanoma in a patient having an ocular tumor, the method comprising
a) administering a PKC inhibitor and administering a c-MET inhibitor to the patient as a neoadjuvant therapy;
b) reducing the size of the ocular tumor by at least 10%; and
c) treating the reduced-size ocular tumor with an interventional procedure.

2. The method of claim 1, wherein the ocular tumor is defined by a set of dimensions comprising (i) greater than or equal to 5 mm largest basal diameter (LBD) and any apical height, or (ii) any LBD and greater than 3 mm apical height.

3. A method of treating a patient having uveal melanoma with an ocular tumor with a size indicated for an interventional procedure comprising enucleation of the eye, the method comprising
a) administering a PKC inhibitor and administering a c-MET inhibitor to the patient as a neoadjuvant therapy;
b) reducing the size of the ocular tumor by at least 10%; and
c) treating the reduced-size ocular tumor with an interventional procedure other than enucleation.

4. The method of claim 3, wherein the ocular tumor is defined by a set of dimensions comprising (i) up to 22 mm in LBD and/or (ii) up to 15 mm in apical height.

5. A method of treating a patient having uveal melanoma with an ocular tumor with a size indicated for an interventional procedure comprising irradiating the ocular tumor, the method comprising
a) administering a PKC inhibitor and administering a c-MET inhibitor to the patient as a neoadjuvant therapy;
b) reducing the size of the ocular tumor by at least 10%; and
c) treating the reduced-size ocular tumor with an interventional procedure other than enucleation.

6. The method of claim 5, wherein the ocular tumor is defined by a set of dimensions comprising (i) at least 6 mm in LBD and/or (ii) at least 3 mm in apical height.

7. The method of claim 1, wherein the interventional procedure comprises local surgical resection of the tumor, irradiating the tumor, or any combination thereof.

8. The method of claim 1, wherein the interventional procedure comprises irradiating the tumor.

9. A method of treating uveal melanoma in a patient having an ocular tumor, the method comprising:
a) determining a first therapeutically effective dose of radiation for treating the ocular tumor;
b) administering a PKC inhibitor and administering a c-MET inhibitor to the patient as a neoadjuvant therapy;
c) reducing the size of the ocular tumor; and
d) irradiating the reduced-size ocular tumor with a second therapeutically effective dose of radiation less than the first therapeutically effective dose of radiation.

10. The method of claim 9, wherein in step (c) the ocular tumor is reduced by at least 10%.

11. The method of claim 9, wherein the ocular tumor before step (a) is defined by a set of dimensions comprising (i) at least 6 mm in LBD and/or (ii) at least 3 mm in apical height.

12. The method of claim 9, wherein in step (d) the second therapeutically effective dose of radiation is at least 10% less than the first therapeutically effective dose of radiation.

13. The method of claim 1, wherein the PKC inhibitor is Compound 1: 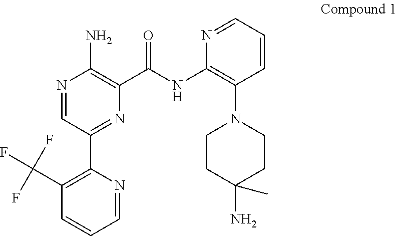 [SMILES: CC1(N)CCN(c2cccnc2NC(=O)c2nc(-c3ncccc3C(F)(F)F)cnc2N)CC1]
or a pharmaceutically acceptable salt thereof.

14. The method of claim 1, wherein the c-MET inhibitor is Compound 2: 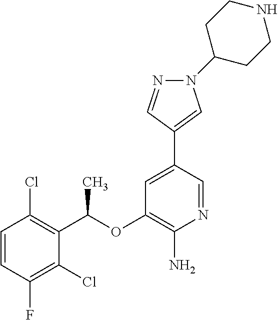 [SMILES: C[C@@H](Oc1cc(-c2cnn(C3CCNCC3)c2)cnc1N)c1c(Cl)ccc(F)c1Cl]
or a pharmaceutically acceptable salt thereof.

15. The method of claim 1, wherein the interventional procedure is a globe preserving treatment (GPT).

16. The method of claim 1, wherein the interventional procedure is selected from the group consisting of plaque brachytherapy (PBT), external beam radiotherapy (EBRT), stereotactic radiosurgery (SRS), and any combination thereof.

17. The method of claim 1, wherein the interventional procedure is plaque brachytherapy (PBT).

18. The method of claim 1, wherein the globe preserving treatment is selected from the group consisting of limited surgical resection or ablation (LSRA), radiation therapy (RT), brachytherapy (BT), external beam radiotherapy (EBRT), stereotactic radiosurgery (SRS), radiation therapy alone (RTA), radiation therapy and supplemental laser therapy (RT+SLT), and any combination thereof.

19. The method of claim 1, wherein the method further comprises continuing administering a PKC inhibitor and administering a c-MET inhibitor to the patient as an adjuvant therapy during the period of the interventional procedure.

20. The method of claim 1, wherein the method further comprises continuing administering a PKC inhibitor and administering a c-MET inhibitor to the patient as an adjuvant therapy after the completion of the interventional procedure.

21. The method of claim 20, wherein the adjuvant therapy is administered at least 4 weeks, at least 5 weeks, or at least 6 weeks after the completion of the interventional procedure.

22. The method of claim 1, wherein the PKC inhibitor and the c-MET inhibitor are both administered orally.

23. The method of claim 1, wherein the method further comprises preserving the eye globe which had the ocular tumor.

24. The method of claim 1, wherein the method further comprises preserving a function of the eye which had the ocular tumor.

25. The method of claim 24, wherein preserving the function of the eye comprises at least partially preserving the eye function.

26. The method of claim 1, wherein the method further comprises preventing irreversible vision loss.

27. The method of claim 1, wherein the method further comprises preserving visual symptoms.

28. The method of claim 1, wherein the method further comprises preserving depth perception.

29. The method of claim 1, wherein the method further comprises preserving vision for greater than at least 1 year.

30. The method of claim 1, wherein the method further comprises preventing metastasis.

31. The method of claim 1, wherein the method further comprises delaying onset of metastasis.

32. The method of claim 1, wherein the ocular tumor is spatially separated from the optic nerve.

33. The method of claim 1, wherein the ocular tumor, prior to administering the neoadjuvant therapy, has a size
(i) from 5 mm to 19 mm LBD and less than 2.5 mm apical height,
ii) less than or equal to 19 mm LBD and 2.5-10 mm apical height,
iii) greater than 19 mm LBD,
iv) greater than 10 mm apical height and any size LBD,
v) greater than 8 mm apical height and the tumor is located close to the optic nerve,
vi) greater than 6 mm and less than 12 mm apical height, and not greater than 16 mm LBD,
vii) greater than 3 mm and less than 8 mm apical height, and not greater than 16 mm LBD,
viii) greater than 8 mm apical height, and greater than 16 mm LBD,
ix) not greater than 16 mm LBD, or
x) greater than 10 mm in apical height.

34. The method of claim 1, wherein the size of the ocular tumor after administering the neoadjuvant therapy but prior to the interventional procedure is reduced by at least 20%.

35. The method of claim 1, wherein the PKC inhibitor, or an equivalent dose of a pharmaceutically acceptable salt thereof, is administered at a dose of about 200 mg to about 400 mg two times per day (BID).

36. The method of claim 1, wherein the PKC inhibitor, or an equivalent dose of a pharmaceutically acceptable salt thereof, is administered at a dose of about 300 mg two times per day (BID).

37. The method of claim 1, wherein the c-MET inhibitor, or an equivalent dose of a pharmaceutically acceptable salt thereof, is administered at a dose of about 200 mg BID.

38. The method of claim 1, wherein the PKC inhibitor, or an equivalent dose of a pharmaceutically acceptable salt thereof, is administered at a dose of about 400 mg to about 600 mg daily; and the c-MET inhibitor, or an equivalent dose of a pharmaceutically acceptable salt thereof, is administered at a dose of about 400 mg to about 500 mg daily.

39. The method of claim 1, wherein the PKC inhibitor, or an equivalent dose of a pharmaceutically acceptable salt thereof and the c-MET inhibitor, or an equivalent dose of a pharmaceutically acceptable salt thereof, are both administered daily for at least 7 days, at least 14 days, at least 21, days, at least 28 days, at least 56 days, at least 84 days, at least, 112 days, at last 140 days, or at least 168 days.

40. The method of claim 1, wherein the uveal melanoma is a solid tumor harboring GNAQ or GNA11 mutations.

41. The method of claim 1, wherein the ocular tumor is intraocular tumor.

42. The method of claim 1, wherein the size of the ocular tumor is measured using RECIST 1.1 criteria.

43. The method of claim 1, wherein the size of the ocular tumor is determined by CT scan, MRI, or ultrasound, or a combination thereof.

44. The method of claim 1, wherein the neoadjuvant therapy is administered to the patient for at least 1 month, at least 2 months, at least 3 months, at least 4 months, at least 5 months, or at least 6 months.

45. The method of claim 1, wherein the adjuvant therapy is administered to the patient for at least 1 month, at least 2 months, at least 3 months, at least 4 months, at least 5 months, or at least 6 months.
